(19) United States
(12) Patent Application Publication
(10) Pub. No.: US 2023/0398088 A1
(43) Pub. Date: Dec. 14, 2023

(54) Lipid Prophylactic Brain Injury Treatment

(71) Applicants: THE TRUSTEES OF THE UNIVERSITY OF PENNSYLVANIA, Philadelphia, PA (US); The United States of America as Represented by the Department of Veterans Affairs, Washington, DC (US)

(72) Inventors: Daniel Kacy Cullen, Media, PA (US); Carolyn E. Keating, Philadelphia, PA (US); Kevin D. Browne, Berlin, NJ (US)

(21) Appl. No.: 18/034,245

(22) PCT Filed: Oct. 29, 2021
(86) PCT No.: PCT/US21/57236

§ 371 Date: Apr. 27, 2023

Related U.S. Application Data
(60) Provisional application No. 63/108,160, filed on Oct. 30, 2020.

(51) Int. Cl. A61K 31/202; A61K 31/688; A61K 9/00; A61K 9/51; A23L 33/12; A61P 25/00

(52) U.S. Cl. CPC A61K 31/202; A61K 31/688; A61K 9/0053; A61K 9/5123; A23L 33/12; A61P 25/00

(57) ABSTRACT

In one aspect, the present disclosure relates to a composition comprising a therapeutically effective amount a polyunsaturated fatty acid (PUFA). In another aspect, the present disclosure relates to a method of reducing a brain injury in a subject having an elevated risk of a traumatic brain injury or concussion, the method comprising: prophylactically administering to the subject a composition comprising a therapeutically effective amount of a PUFA. In yet another aspect, the present disclosure relates to sports drinks and dietary supplements comprising a PUFA. In some embodiments, the PUFA is an omega-6 PUFA such as omega-6 docosapentaenoic acid.

### CROSS-REFERENCE TO RELATED APPLICATIONS

This application claims priority under 35 U.S.C. § 119(e) to U.S. Provisional Patent Application No. 63/108,160, filed Oct. 30, 2020, the disclosure of which is incorporated herein by reference in its entirety.

### BACKGROUND OF THE INVENTION

Traumatic brain injury (TBI) is caused by a discrete physical event that transmits forces to the brain. These forces can directly damage cells in what is known as the primary injury. Mild TBI, such as concussions, can cause disabilities without any apparent immediate brain cell death, suggesting that neurons survive but become dysfunctional after injury. One way that neurons can become injured is through damage to their plasma membrane, a wall of lipids and proteins that separate the inside of the cell from the outside environment. Indeed, the plasma membrane of neurons has been shown to be directly vulnerable in TBI. Specifically, this damage can occurs through micro- or nano-sized tears in the plasma membrane of cells, termed permeability or mechanoporation. These tears are often transient, and many damaged cells survive. However, these cells can display functional alterations or delayed death due to a loss of ionic and osmotic homeostasis and disruption of electrokinetic transport resulting from the transient loss of plasma membrane integrity.

The response of the plasma membrane to applied forces—i.e., whether it bends or breaks—depends on its mechanical properties. Materials that are more rigid or stiff are expected to tear when they experience the rapid application of forces that occur during TBI, while materials that are more elastic or flexible are expected to bend. Thus, the response to injurious forces may be modified by changing the mechanical properties of the plasma membrane. The extent of pathophysiology and neurological dysfunction associated with TBI may be reduced if a method was devised to render the cell membrane of neurons less susceptible to damage.

There is a need in the art for a composition that can reduce damage to the brain plasma membrane sustained during a concussion or TBI as well as a methods of using the composition. The present invention addresses these unmet needs.

### SUMMARY OF THE INVENTION

In one aspect, the invention provides a method of reducing a brain injury in a subject having an elevated risk of a traumatic brain injury (TBI) or concussion, the method comprising: prophylactically administering to the subject a composition comprising a therapeutically effective amount of a polyunsaturated fatty acid.

In another aspect, the invention provides a sports drink comprising a polyunsaturated fatty acid composition encapsulated in a lipid nanoparticle, wherein the sports drink further comprises sugar and an electrolyte.

In another aspect, the invention provides a dietary supplement comprising a polyunsaturated fatty acid composition encapsulated in a lipid nanoparticle.

In some embodiments, the composition comprising the polyunsaturated fatty acid is encapsulated in a lipid nanoparticle.

In some embodiments, the polyunsaturated fatty acid is an omega-6 polyunsaturated fatty acid.

In some embodiments, the omega-6 polyunsaturated fatty acid is omega-6 docosapentaenoic acid.

In some embodiments, the composition further comprises cholesterol, a triglyceride, a sphingolipid, or a combination thereof.

In some embodiments, the composition comprises a sphingomyelin.

In some embodiments, the composition comprises an ester derived from glycerol and three fatty acids selected from the group consisting of myristic acid, palmitic acid, stearic acid, palmitoleic acid, oleic acid, linoleic acid, α-linoleic acid, vaccenic acid, and combinations thereof.

In some embodiments, the lipid nanoparticle comprises an omega-6 polyunsaturated fatty acid, cholesterol, a sphingomyelin, or a combination thereof.

In some embodiments, the method further comprises the step of continuing to administer to the subject the composition comprising a therapeutically effective amount of a polyunsaturated fatty acid after the subject has suffered a TBI or concussion.

In some embodiments, the composition is orally administered to the subject.

In some embodiments, the method reduces formation of micro- or nano-sized tears in a plasma membrane of a brain cell of the subject formed during a concussion or TBI primary injury.

In some embodiments, the sports drink comprises a sphingomyelin.

In some embodiments, the sports drink comprises an ester derived from glycerol and three fatty acids selected from the group consisting of myristic acid, palmitic acid, stearic acid, palmitoleic acid, oleic acid, linoleic acid, α-linoleic acid, vaccenic acid, and combinations thereof.

In some embodiments, the dietary supplement comprises a sphingomyelin.

In some embodiments, the dietary supplement comprises an ester derived from glycerol and three fatty acids selected from the group consisting of myristic acid, palmitic acid, stearic acid, palmitoleic acid, oleic acid, linoleic acid, α-linoleic acid, vaccenic acid, and combinations thereof.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 depicts a timeline of the experiments. All animals were fed diets for 4 weeks. The Lipid Analysis group was sacrificed after feeding to assess changes in brain phospholipid content. The Acute and 7d groups received FPI or sham injuries after feeding. For the acute group, on the day of injury animals were injected with a cell impermeability dye (Lucifer Yellow, LY) two hours prior to injury. Immediately after injury they were sacrificed for histological analysis of permeability and inflammation. For the 7d group, animals did not receive LY injections but instead were survived for 1 week after injury, at which time they were sacrificed for histological analysis of lesion size, inflammation, and NeuN reactivity.

FIG. 2 depicts the diet formulation details. Provided from Envigo Teklad.

FIGS. 3A-3D depict that injury level and body weight do not vary among groups. One-way ANOVA revealed that the average injury level was no different among animals receiving different diets in the acute group (FIG. 3A; p=0.1913) or the 7 day survival group (FIG. 3B; p=0.3046). FIG. 3C: In the acute group, two-way ANOVA revealed no significant differences in body weight at injury between injury groups (p=0.3780), diet (p=0.9917), or due to their interaction (p=0.8403). FIG. 3D: In the 7 day survival group, two-way ANOVA revealed no significant differences in body weight due to diet (p=0.1757), time (at injury or at sacrifice 7 days later; p=0.9206), or their interaction (p=0.9833). Error bars±SEM. depict in vivo cerebral microvascular permeability.

FIGS. 4A-4B depict that diets altered brain fatty acid content. FIG. 4A: Fish Oil decreased the percentage of saturated fatty acids and increased the percentage of unsaturated fatty acids. FIG. 4B: Analysis of specific fatty acids revealed that Fish Oil increased DHA, other MUFAs, and other PUFAs, and decrease other SFAs compared to the other diets. High Fat increased omega-6 DPA. Error bars±SEM. Lipid notation: C (number of carbon atoms): (number of double bonds); n3, -6, or -9 (omega-3, -6, or -9) refers to location of first double bond from methyl end. Abbreviations: AA, arachidonic acid; DHA, docosahexaenoic acid; DPA, docosapentaenoic acid; DTA, docosatetraenoic acid; MUFA, monounsaturated fatty acid; PUFA, polyunsaturated fatty acid; SFA, saturated fatty acids; UFA, unsaturated fatty acids. P values: <0.05, *; <0.01, **; <0.001, ***; <0.0001, ****.

FIG. 5 depicts the P values for fatty acid comparisons between diet groups. (ns, not significant).

FIGS. 6A-6E depict that diets altered neuronal permeability. FIG. 6A: Representative image of cortical region analyzed. Scale bar 250 μm. FIG. 6B: Example of permeabilized neurons in the medial cortex. Scale bar 100 μm. FIG. 6C: Representative images of the number and extent of permeabilized neurons in the ipsilateral medial cortex from each group. Scale bars 1 mm. FIG. 6D: Global quantification of the number of permeabilized neurons per mm². Two-way ANOVA revealed a significant injury effect (p<0.0001), but no effect of diet (p=0.7101) or the interaction term (p=0.9576). Multiple comparisons revealed a significant difference between sham and injured animals for each diet (Control p=0.0112, High Fat p=0.0017, Fish Oil p=0.0026). Error bars±SEM. FIG. 6E: Quantification of the number of permeabilized neurons per mm² in the ipsilateral medial cortex. Two-way ANOVA revealed a significant effect of diet (p=0.0492) and interaction (p=0.0389), but not injury (p=0.2094), likely due to the large number of permeabilized cells observed in Fish Oil shams. Sham animals fed Fish Oil had significantly more permeabilized neurons than sham animals fed Control (p=0.0405) or High Fat (p=0.0433) diets. Error bars±SEM.

FIGS. 7A-7B depict that increased permeability was layer-specific. FIG. 7A: Representative image of layer subdivisions in the medial cortex. Scale bar 1 mm. FIG. 7B: Quantification of permeabilized neurons per layer. Two-way ANOVA revealed a significant effect of layer (p<0.0001), diet and injury together (p=0.0130), and their interaction (p<0.0008). In sham animals, the increased permeability observed in animals fed Fish Oil was mainly restricted to layer 5. Injury increased permeabilized cells in layer 6. Multiple comparisons: Layer 6 Ctrl sham vs. Ctrl injured, p=0.0187; Ctrl injured layer 1 vs. layer 6, p=0.0182; Ctrl injured layers 2-4 vs. layer 6, p=0.0407; HF injured layer 1 vs. layer 6, p=0.0144; HF injured layers 2-4 vs. layer 6, p=0.0327; FO sham layer 1 vs. layer 5, p=0.0393. Error bars±SEM.

FIGS. 8A-8G depict that diets altered the intensity of dye uptake by permeabilized neurons. FIGS. 8A-8C: Representative images of low (FIG. 8A, arrows), medium (FIG. 8B), and high (FIG. 8C) intensity LY uptake. Scale bars 50 μm. FIG. 8D: Quantification of average neuronal LY intensity in arbitrary units (AU). Two-way ANOVA revealed a significant effect of injury (p=0.0048), a nearly significant effect of diet (p=0.0554), and no interaction (p=0.1501). Tukey's multiple comparisons tests showed an injury-induced increase in intensity in animals fed the Control diet (p=0.0290), while injured animals fed the High Fat diet had less intense permeability than injured animals fed the Control diet (p=0.0465). Error bars±SEM. FIGS. 8E-8F: Histograms illustrating the relative frequency distributions of cell intensity for sham (FIG. 8E) and injured (FIG. 8F) animals. FIG. 8G: Relative frequency distributions of individual experimental groups. Lines of best fit were determined using one-phase exponential decay equations, the accuracy of which were confirmed using standard error of the residuals (Sy.x). Extra sum-of-squares F tests were used to compare the nonlinear regression curves within sham and injured groups separately. If one curve did not adequately fit all three datasets within each group, the test was run again with two curves at a time to determine which curves were different; for these analyses, the cutoff value of significance was adjusted by dividing p=0.05 by the number of comparisons (3), to produce a new cutoff value of p=0.01667. For shams, one curve did not fit all datasets (p=0.0055), with the animals fed the Fish Oil diet displaying an intensity distribution with more intense dye uptake than animals fed the Control (p=0.0054) or High Fat (p=0.0028) diets. For injured animals, one curve also did not fit all datasets (p<0.0001), with animals fed the High Fat diet exhibiting more cells with less intense dye uptake than animals fed the Control or Fish Oil diets (p=0.0001 for both comparisons).

FIGS. 9A-9E depict that diets did not alter inflammation in the medial cortex acutely. FIG. 9A: Representative images of astrocyte (GFAP) and microglia (Iba1) reactivity in sham and injured animals. Scale bars 50 μm. FIGS. 9B-9C: Quantification of astrocyte reactivity (GFAP density (FIG. 9B) and intensity (FIG. 9C)). Two-way ANOVA revealed no significant effects of diet, injury, or their interaction, though there appears to be a weak trend for injured animals fed the Fish Oil diet to have less astrocyte reactivity. FIGS. 9D-9E: Quantification of microglia reactivity (Iba1 density (FIG. 9D) and intensity (FIG. 9E)). Two-way ANOVA revealed no significant effects of diet, injury, or their interaction. Error bars±SEM.

FIGS. 10A-10D: depict that the High Fat diet reduced the lesion area. FIGS. 10A-10C: Representative H&E images from anterior sections of animals fed Control (FIG. 10A), High Fat (FIG. 10B), or Fish Oil (FIG. 10C). Dashed circles highlight lesion. Scale bars 2 mm. FIG. Quantification of lesion area across anterior (bregma −3.2 to −4.0 mm), posterior (bregma −5.0 to −6.2 mm), and summed brain sections. Animals fed the High Fat diet had significantly smaller lesion areas in anterior sections (p=0.0216) and when sections are summed (p=0.0361), and trended towards significantly smaller areas in posterior sections (p=0.0879). Error bars±SEM.

FIGS. 11A-11D depict inflammation and NeuN in and around the lesion 7 days after injury. Representative images of GFAP (FIG. 11A), Iba1 (FIG. 11B), NeuN (FIG. 11C), and merge (FIG. 11D). The gray matter cortical lesion (as defined by H&E), as well as the peri-lesion gray matter 1 mm around the lesion, were quantified. Scale bar 1 mm.

FIGS. 12A-12L depict that diets may alter NeuN expression and inflammation in and around the lesion 7 days after injury. The gray matter lesion was defined using H&E, and the peri-lesion area consisted of the gray matter 1 mm surrounding the lesion. Animals with only a white matter lesion, or no lesion datable via H&E, were excluded. Quantification of NeuN density and intensity in the lesion (FIG. 12A, FIG. 12D) and peri-lesion (FIG. 12G, FIG. 12J) regions revealed a very weak trend for increased NeuN density in the lesion of injured animals fed the High Fat diet (p=0.3684) and strong trend for decreased NeuN density in the peri-lesion area of injured animals fed Fish Oil (p=0.0769). Analysis of GFAP density and intensity in the lesion (FIG. 12B, FIG. 12E) and peri-lesion (FIG. 12H, FIG. 12K) areas showed no significant differences in astrocyte reactivity between diets. Assessment of Iba1 density and intensity in the lesion (FIG. 12C, FIG. 12F) and peri-lesion (FIG. 12I, FIG. 12L) regions resulted in a weak trend for increased microglia density in the lesion of injured animals fed the High Fat diet (p=0.2122) and a strong trend for increased intensity (p=0.0734); while in the peri-lesion area there were weak trends for injured animals fed Fish Oil to display decreased Iba1 reactivity (density, p=0.2400; intensity, p=0.2271). Error bars±SEM.

### DETAILED DESCRIPTION OF THE DISCLOSURE

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, selected methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “composition” or “pharmaceutical composition” refers to at least one compound useful within the invention that is optionally mixed with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

A “disease” is a state of health of subject wherein the subject cannot maintain homeostasis, and wherein if the disease is not ameliorated then the subject's health continues to deteriorate. In contrast, a “disorder” in an subject is a state of health in which the subject is able to maintain homeostasis, but in which the subjects's state of health is less favorable than it would be in the absence of the disorder. Left untreated, a disorder does not necessarily cause a further decrease in the subject's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein the term “polyunsaturated fatty acid” refers to a type of unsaturated fatty acid with more than one double bond. Because of the presence of several double bonds, the polyunsaturated fatty acids tend to have higher boiling point than the monounsaturates (which only have one double bond). Similar to other unsaturated fatty acids, the polyunsatured fatty acids are liquids at room temperature. They include the essential fatty acids, omega-3 fatty acid, omega-6 fatty acid, and linoleic acids. They are found chiefly in fish, seeds, bananas, nuts, and vegetable oils.

The terms “patient,” “subject,” or “individual” are used interchangeably herein, and refer to any animal, or cells thereof whether in vitro or in situ, amenable to the methods described herein. In a non-limiting embodiment, the patient, subject, or individual is a human.

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about % to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Description

The present invention provides in one aspect a composition comprising an omega-6 polyunsaturated fatty acid (PUFA). In some embodiments, the omega-6 PUFA comprises a chain of 16 or more carbon atoms. In one embodiment, the omega-6 PUFA is omega-6 docosapentaenoic acid. In some embodiments, the composition further comprises cholesterol, a triglyceride, a sphingolipid, or a combination thereof. The composition may further comprise a pharmaceutical carrier or an inactive ingredient. The present invention also provides a method a of reducing a brain injury in a subject having an elevated risk of a traumatic brain injury (TBI) or concussion, the method comprising: prophylactically administering to the subject a composition comprising a therapeutically effective amount of a polyunsaturated fatty acid. In one embodiment, the PUFA is an omega-6 PUFA comprising a chain of 16 or more carbon atoms such as omega-6 docosapentaenoic acid. In some embodiments, administration of a PUFA reduces injury to the brain following a TBI or concussion by changing the mechanical properties of the plasma membrane, leading to a reduction in the incidence of micro- or nano-sized tears in the plasma membrane of the brain cells during the primary brain injury. In some embodiments, administration of a PUFA increases the elasticity of the plasma membrane of the brain cells while not significantly changing the permeability. In yet another aspect, the present invention relates to an edible product or a pharmaceutical composition that can administered to the subject prophylactically to reduce brain injury associated with a TBI or concussion.

### Compositions

In one aspect, the present invention relates to a composition comprising a PUFA. In some embodiments, the PUFA is an omega-3 fatty acid. Exemplary omega-3 fatty acids include, but are not limited to, all-cis-7,10,13-hexadecatrienoic acid (hexadecatrienoic acid), all-cis-9,12,15-octadecatrienoic acid (α-linolenic acid), all-cis-6,9,12,15-octadecatetraenoic acid (stearidonic acid), all-cis-11,14,17-eicosatrienoic acid (eicosatrienoic acid), all-cis-8,11,14,17-eicosatetraenoic acid (eicosatetraenoic acid), all-cis-5,8,11,14,17-eicosapentaenoic acid (eicosapentaenoic acid), all-cis-6,9,12,15,18-heneicosapentaenoic acid (heneicosapentaenoic acid), all-cis-7,10,13,16,19-docosapentaenoic acid (docosapentaenoic acid, clupanodonic acid), all-cis-4,7,10,13,16,19-docosahexaenoic acid (docosahexaenoic acid), all-cis-9,12,15,18,21-tetracosapentaenoic acid (tetracosapentaenoic acid), all-cis-6,9,12,15,18,21-tetracosahexaenoic acid (tetracosahexaenoic acid, nisinic acid), and combinations thereof. In some embodiments, the PUFA is an omega-6 fatty acid. Exemplary omega-6 fatty acids include, but are not limited to, all-cis-9,12-octadecadienoic acid (linoleic acid), all-cis-6,9,12-octadecatrienoic acid (gamma-linolenic acid), 8E,10E,12Z-octadecatrienoic acid (calendic acid), all-cis-11,14-eicosadienoic acid (eicosadienoic acid), all-cis-8,11,14-eicosatrienoic acid (dihomo-gamma-linolenic acid), all-cis-5,8,11,14-eicosatetraenoic acid (arachidonic acid), all-cis-13,16-docosadienoic acid (docosadienoic acid), all-cis-7,10,13,16-docosatetraenoic acid (adrenic acid), all-cis-4,7,10,13,16-docosapentaenoic acid (osbond acid), all-cis-9,12,15,18-tetracosatetraenoic acid (tetracosatetraenoic acid), all-cis-6,9,12,15,18-tetracosapentaenoic acid (tetracosapentaenoic acid), and combinations thereof. In some embodiments, the composition comprises both an omega-3 fatty acid and an omega-6 fatty acid. In one embodiment, the composition comprises an omega-6 fatty acid having a carbon chain with 16 or more carbon atoms. In one embodiment, the composition comprises omega-6 docosapentaenoic acid. In another embodiment, the composition comprises omega-6 docosapentaenoic acid as well as eicosapentaenoic acid and/or docosahexaenoic acid.

In some embodiments, the composition further comprises cholesterol. In one embodiment, the composition comprises milkfat. In one embodiment, the composition comprises a triglyceride. In some embodiments, the triglyceride is composed of glycerol and three fatty acids including, but not limited to, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, and combinations thereof. In one embodiment, the composition comprises a sphingolipid. Exemplary types of sphingolipids include, but are not limited to, sphingomyelins, ceramides, phytoceramide, glycosphingolipids, gangliosides, cerebrosides, sulfatides, and combinations thereof.

In one embodiment, the composition comprises an additional ingredient known or believed by a person of skill in the art to be beneficial in treating or preventing brain injury. In one embodiment, the brain injury is an injury following a TBI or a concussion. In some embodiments, the additional ingredient is a pain reliever, an anti-anxiety medication, an anticoagulant, an anticonvulsant, an antidepressant, a muscle relaxant, a stimulant, an anti-inflammatory, or combinations thereof. In one embodiment, the additional ingredient is curcumin, turmeric, resveratrol, or a combination thereof.

In some embodiments, the composition comprises an organic or aqueous solvent. In certain embodiments, the composition comprises an inactive ingredient. The inactive ingredient may be any inactive ingredient known to a person of skill in the art. In certain embodiments, the inactive ingredient is selected from the group consisting of excipients, diluents, fillers, binders, disintegrants, lubricants, colorants, preservatives, stabilizers, viscosity increasing agents, sweeteners, flavoring agents, and any combinations thereof.

In one embodiment, the PUFA composition is encapsulated in a nanoparticle. In one embodiment, the nanoparticle is a lipid nanoparticle (LNP). The LNP can comprise any components known to a person of skill in the art for the formation of lipid nanoparticles. In some embodiments, the LNP comprises a triglyceride, a diglyceride, a monoglyceride, a fatty acid, a steroid, a wax, an emulsifier, or combinations thereof. In one embodiment, the LNP comprises soybean oil. In some embodiments, the LNP comprises one or more of the lipid components of the PUFA composition including, but not limited to, a polyunsaturated fatty acid, cholesterol, a triglyceride, a sphingolipid, or a combination thereof. In one embodiment, the LNP comprises an omega-6 PUFA, cholesterol, a sphingomyelin, or a combination thereof. Therefore, in some embodiments both the vehicle (LNP itself) and the cargo (PUFA composition) comprise lipid components of the PUFA composition.

The LNP encapsulating the PUFA composition can have any size known to a person of skill in the art. In one embodiment, the LNP containing cargo (PUFA composition) is between about 0.5 nm to about 3000 nm in diameter. In one embodiment, the LNP is between about 0.5 nm to about 2500 nm in diameter. In one embodiment, the LNP is between about 0.5 nm to about 2000 nm in diameter. In one embodiment, the LNP is between about 0.5 nm to about 1500 nm in diameter. In one embodiment, the LNP is between about 0.5 nm to about 1000 nm in diameter. In one embodiment, the LNP is between about 0.5 nm to about 800 nm in diameter. In one embodiment, the LNP is between about 0.5 nm to about 600 nm in diameter. In one embodiment, the LNP is between about 0.5 nm to about 400 nm in diameter. In one embodiment, the LNP is between about 0.5 nm to about 200 nm in diameter. In one embodiment, the LNP is between about 0.5 nm to about 50 nm in diameter. In one embodiment, the LNP is between about 2 nm to about 20 nm in diameter.

### Methods

In another aspect, the present invention relates to a method of reducing a brain injury in a subject having an elevated risk of a traumatic brain injury (TBI) or concussion, the method comprising: prophylactically administering to the subject a composition comprising a therapeutically effective amount of a polyunsaturated fatty acid.

In one embodiment, the method reduces direct damage to the cells of the brain in the TBI or concussion primary injury. Although not wishing to be limited by theory, it is believed that the method reduces TBI or concussion primary injury by “shifting the tolerance curve,” for instance, causing an impact that would have caused a mild concussion to be a non-injury, and making an impact that would have been a severe concussion into a mild concussion. In some embodiments, the method reduces secondary brain injury caused by TBI or concussion. The subject can be any subject believed to have an elevated risk of a TBI or concussion. Exemplary subjects include, but are not limited to, the elderly or other individuals prone to falls, soldiers, athletes, police officers, or individuals who have a history of at least one incidence of sustaining a brain injury such as a TBI or concussion.

The polyunsaturated fatty acid can be any PUFA described elsewhere herein. In one embodiment, the PUFA composition is encapsulated in a lipid nanoparticle. In one embodiment, the PUFA is an omega-6 fatty acid having a carbon chain with 16 or more carbon atoms. In one embodiment, the PUFA is omega-6 docosapentaenoic acid. In some embodiments, the composition comprising a PUFA comprises an additional active ingredient. Exemplary active ingredients are described elsewhere herein. In some embodiments, the composition comprising a PUFA comprises an inactive ingredient. Exemplary inactive ingredients are described elsewhere herein. In one embodiment, the composition further comprises cholesterol.

The composition comprising the PUFA can be administered to the subject using any method known to a person of skill in the art. In some embodiments, the composition is administered orally. In one embodiment, the composition is dispersed in a food or beverage which is administered orally. Exemplary food or beverage products are described elsewhere herein. In another embodiment, the PUFA composition or the LNP-PUFA composition is mixed with a pharmaceutically acceptable carrier to form a pharmaceutical composition. Exemplary pharmaceutical compositions and pharmaceutically acceptable carriers are described elsewhere herein. In some embodiments, the pharmaceutical composition is administered orally as a liquid, syrup, pill, tablet, capsule, or gelcap.

The composition can be administered to the subject at any dosage known to a skilled artisan to be safe for administration to a mammal and to provide the desired therapeutic effect. The composition can be administered to the subject any time before the subject engages in an activity that may result in a TBI or concussion. In some embodiments, the composition is administered to the subject at regular intervals (e.g. daily, multiple times a day, once a week, biweekly, every two weeks, every three weeks, monthly). In one embodiment, the composition is administered to the subject daily or every other day over the period of a few weeks or a few months before the subject engages in an activity that may result in a TBI or concussion. In some embodiments, the composition is administered to the subject between about 24 hours and about 1 hour before the subject engages in an activity that may result in a TBI or concussion. In embodiments wherein the subject engages in an activity that may result in a TBI or concussion regularly, the composition can be administered to the subject at regular intervals for an extended period (e.g. for several months, a year, several years).

In some embodiments, the step of prophylactically administering a composition comprising a polyunsaturated fatty acid to the subject provides treatment for a TBI or concussion by reducing the primary brain injury associated with the TBI or concussion. In some embodiments, the primary brain injury associated with a TBI or concussion forms micro- or nano-sized tears in the plasma membrane of the brain cells. While not wishing to be limited by theory, administration of the PUFA may alter the membrane lipid composition in the subject's brain. In some embodiments, the PUFA administered to the subject preferably increases the plasma membrane elasticity without significantly compromising the permeability. While not wishing to be limited by theory, omega-6 PUFAs may increase plasma membrane elasticity without comprising the permeability whereas omega-3 PUFAs may increase both plasma membrane elasticity and permeability. It is hypothesized that fewer unsaturations in an omega-6 PUFA may permit closer packing of the PUFA hydrocarbon chain when compared to an omega-3 PUFA of identical carbon chain length, and thus may not significantly impact permeability. This change in the mechanical properties of the plasma membrane is hypothesized to reduce the incidence of micro- or nano-sized tears in the plasma membrane of the brain cells, and thus reduce the primary injury following a TBI or concussion.

In embodiments wherein the subject suffers a TBI or concussion, the method may further comprise the step of administering to the subject the composition comprising a therapeutically effective amount of a polyunsaturated fatty acid. The PUFA can be any PUFA disclosed elsewhere herein. In some embodiments, the PUFA is an omega-6 PUFA. In one embodiment, the PUFA is omega-6 docosapentaenoic acid. In one embodiment wherein the subject suffers a TBI or concussion, the PUFA is administered as soon as possible after the TBI or concussion and may continue to be administered following the initial administration (e.g. for several days, weeks, or months after the TBI or concussion).

In embodiments wherein the subject suffers a TBI or concussion, the method may further comprise the step of administering to the subject an additional treatment known or believed to be beneficial in treating or preventing brain injury following a concussion or TBI. The additional treatment can be any treatment known to a person of skill in the art to reduce secondary injury and/or improve patient outcomes following a TBI or concussion. Exemplary additional treatments include, but are not limited to, surgery, medication (e.g. pain relievers, anti-anxiety medications, anticoagulants, anticonvulsants, antidepressants, muscle relaxants, stimulants), rehabilitation therapy (e.g. physical therapy, occupational therapy, speech therapy, psychological counseling, vocational counseling, cognitive therapy), and combinations thereof. The additional treatment can be administered before, after, or concurrent with the PUFA or LNP-PUFA composition.

### Edible Product

In yet another aspect, the present invention relates to an edible product comprising the PUFA composition or the lipid nanoparticle containing the PUFA (LNP-PUFA) composition. The PUFA can be any PUFA described elsewhere herein. In one embodiment, the PUFA is an omega-6 PUFA. In one embodiment, the PUFA is omega-6 docosapentaenoic acid.

In one embodiment, the edible product comprises the PUFA composition or LNP-PUFA composition dispersed in a food or beverage. In one embodiment, the PUFA composition or the LNP-PUFA composition is dissolved/dispersed in water. In another embodiment, the PUFA composition or the LNP-PUFA composition is dissolved/dispersed in a sports drink. In some embodiments, the sports drink comprising the PUFA or LNP-PUFA composition further comprises a carbohydrate, an electrolyte, a mineral, a vitamin, a protein, caffeine, or a combination thereof. In one embodiment, the sports drink comprises sodium, potassium, sugar, and may further comprise vitamin A, vitamin B12, vitamin C, and/or caffeine.

In one embodiment, the sports drink comprises an LNP vehicle comprising an omega-6 PUFA, cholesterol, a sphingomyelin, or a combination thereof wherein the LNP vehicle encapsulates a PUFA composition cargo comprising an omega-6 PUFA and a sphingomyelin. In some embodiments, the omega-6 PUFA is omega-6 docosapentaenoic acid. In one embodiment, the sports drink further comprises sugar, an electrolyte, and vitamin B12.

In another embodiment, the edible product is a dietary supplement comprising the PUFA composition or the LNP-PUFA composition. In some embodiments, the dietary supplement is a liquid, syrup, pill, tablet, capsule, or gelcap comprising the PUFA or LNP-PUFA composition. In some embodiments, the dietary supplement comprises a pharmaceutically acceptable carrier.

### Administration/Dosage/Formulations

The invention also encompasses pharmaceutical compositions comprising a PUFA or LNP-PUFA and methods of their use. These pharmaceutical compositions may comprise an active ingredient (which can be one or more compounds of the invention, or pharmaceutically acceptable salts thereof) optionally in combination with one or more pharmaceutically acceptable agents. The compositions set forth herein can be used alone or in combination with additional compounds to produce additive, complementary, or synergistic effects.

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated herein. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated herein. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect, and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated herein.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physician taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In certain embodiments, the dose of a compound of the invention is from about 1 mg and about 2,500 mg. In other embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in other embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated herein.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present invention depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Experimental Examples

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Dietary Manipulation of Vulnerability to Traumatic Brain Injury-Induced Neuronal Plasma Membrane Permeability

### Materials and Methods

### Animals

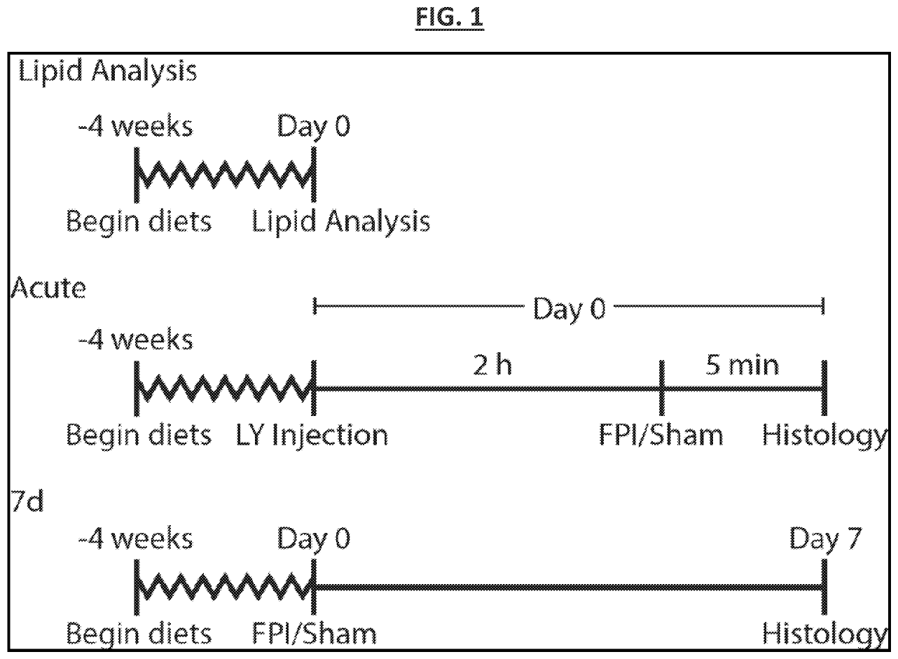

Ninety-four male Sprague Dawley rats aged 8 weeks weighing 250-260 g at the beginning of experimental timeline were used in this study. Pair-housed rats were fed one of three diets (see below) with food and water available ad libitum for 4 weeks. Animals were separated into three groups: lipid analysis (n=12), acute permeability (n=34), and 7 day survival (n=48) (FIG. 1). Animals in the 7 day survival group continued to receive their specific diets during the week after injury.

### Diets

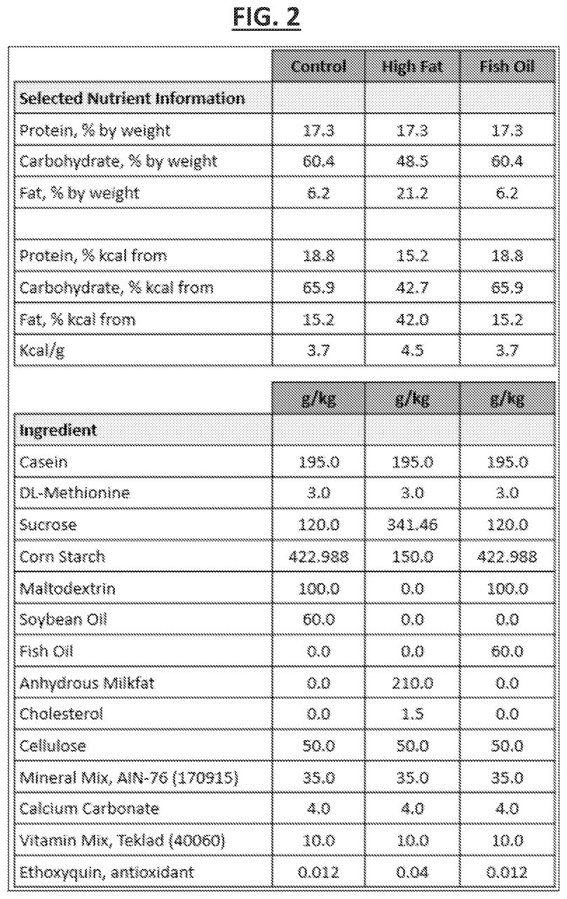

Purified diets were formulated with the help of a nutritionist and purchased through Envigo Teklad. Formulation details can be found in FIG. 2. Briefly, a low fat diet was used as the base for the Control and Fish Oil diets; the Fish Oil diet was supplemented with 6% menhaden fish oil (TD.180920) and the Control diet with 6% soybean oil (TD.180919) to account for the oil content of the fish oil diet. The High Fat diet consisted of the “Western Diet” TD.88137 Adjusted Calories Diet (42% from fat), which included 0.2% total cholesterol, saturated fat >60% total fat, and high sucrose. This formula originated with researchers at Rockefeller University and is used primarily with genetically manipulated mouse models that are susceptible to atherosclerosis, and is also used to study diet-induced obesity, diabetes, and metabolic syndrome. Regardless of diet, each cage of animals was given approximately 420 g of food each week.

### Brain Lipid Analysis

The fatty acid composition of uninjured rat brains after 4 weeks of feeding was analyzed. Four rats from each diet group were euthanized with CO₂ and brains were quickly removed. A 2 mm mid-coronal section weighing approximately 150 mg was taken from each animal. A Fatty Acid Extraction Kit, Low Standard (150 μg/mL) (Sigma, St. Louis, MO) using chloroform and methanol was used for lipid extraction. 100 μL of the total lipid extract was used for fatty acid transesterification. After drying the samples under nitrogen, 1% H₂SO₄ (Sigma-Aldrich, St. Louis, MO) in methanol (EMD Millipore Burlington, MA) and hexane (Sigma-Aldrich, St. Louis, MO) were added and heated at 70° C. for 3 hours. Once cooled to room temperature, hexane and 5% NaCl (Sigma, St. Louis, MO) were added and samples were vortexed before centrifuging at 500×g for 5 minutes. After centrifugation, the top hexane layer was transferred to a clean glass tube and dried under nitrogen. The transesterified lipids were reconstituted with 65 μL of hexane and stored at −20° C. in gas chromatography vials layered with nitrogen until analysis.

Gas chromatography/mass spectrometry (GC/MS) was used to analyze brain fatty acid methyl esters (FAMEs). Samples were run on an Agilent 5973 GCMS (Agilent, Wilmington, DE) using electron ionization. Samples were injected (inlet temp 220° C., split 5:1) onto a nonpolar HP-5MS column (30 m×0.25 mm×0.25 μm; Agilent, Wilmington, DE) with helium as a carrier gas at a constant flow of 1 mL/min. The initial temperature gradient started at 100° C., increasing at 4° C./min to 220° C. and was then held for 12 min. Next, the gradient increased at 2° C./min to 240° C. and was held for 8 min. The Mass Selective Detector (MSD) operated at 70 eV. The Source temp was 230° C., the Quad temp 150° C., and the Interface temp 225° C. All detectable FAMEs were confirmed using Supelco 37 Component FAME mix (Supelco, Sigma-Aldrich, Bellefonte, PA) as a reference. Nonadecanoic acid ethyl ester included in the Fatty Acid Extraction Kit was used as the internal standard control. Hexane was used as blank in the experiment. Agilent ChemStation software was used to compare peaks to the NIST mass spectral library.

### Lucifer Yellow (LY) Intracerebroventricular Injection

The cell impermeable dye Lucifer Yellow (LY; Invitrogen, Carlsbad, CA) was used to label cells for acute loss of membrane integrity. On the day of injury, rats were anesthetized with isoflurane (Phoenix, St. Joseph, MO) and placed in a stereotaxic frame. Under aseptic conditions, bupivacaine (0.5 mg/kg; Hospira, Lake Forest, IL) was administered subcutaneously along the scalp and an incision was made to visualize bregma. A craniectomy was made 0.8 mm posterior, 1.8 mm lateral (on the right-hand side) to bregma. A 10 μL Hamilton syringe (Hamilton, Reno, NV) was lowered 3.4 mm deep from the surface of the brain. After stabilizing for 2 minutes, 10 μL of LY at a concentration of 10 mg/mL in 0.9% sterile saline (Baxter, Deerfield, IL) was delivered into one ventricle at a rate of 2 μL/min using an UltraMicroPump III (World Precision Instruments, Sarasota, FL). Once LY delivery was complete, the needle was again allowed to stabilize for two minutes before being slowly withdrawn from the brain. LY was allowed to circulate throughout the brain for two hours prior to injury.

### Injury

After 4 weeks of feeding, rats were subjected to lateral fluid percussion injury (FPI) or sham injuries. Rats in the 7d survival group that did not receive LY injections were anesthetized with isoflurane and placed in a stereotaxic frame. Under aseptic conditions, bupivacaine (0.5 mg/kg) was administered subcutaneously along the scalp. Rats in the acute permeability group that had received LY injections were prepared for FPI injury immediately after LY delivery and remained under anesthesia. A 5 mm craniectomy was made on the left side of the skull above the hippocampus. The female end of a Luer-lok (BD, Franklin Lakes, NJ) with an internal diameter of 3.5 mm acted as the injury hub. The hub was attached to the craniectomy using dental cement (Densply, York PA) and filled with 0.9% saline, and rats were allowed to recover from anesthesia. Two hours after LY injection for the acute permeability group or 1 hour after craniectomy for the 7 day survival group, rats were briefly re-anesthetized until no pinch reflex was observed, and the injury hub was attached to the FPI device (Virginia Commonwealth University Biomedical Engineering, Richmond, VA). Once whisking behavior was observed, the device pendulum was dropped to deliver a fluid pulse to the exposed dura. The average injury level for rats in the acute permeability group was 2.26±0.069 atm (±standard deviation (SD); min 2.16 atm, max 2.38 atm). The average injury level for rats in 7 day survival group was 2.20±0.046 atm (±SD; min 2.11 atm, max 2.32 atm). Immediately after injury, the presence of apnea, seizure, and hematoma were assessed. There was no mortality resulting from injury. Animals in the 7 day survival group had the injury hub and dement cement removed immediately after injury, and the scalp incision was stapled closed. These animals received intraperitoneal buprenorphine (0.05 mg/kg, Reckitt Benckiser, Parsippany, NJ) and recovery was monitored daily until sacrifice. Sham animals underwent the same procedures, except the pendulum was not dropped while the rats were attached to the FPI device. All rats were weighed before injury, and in the case of the 7 day survival group, before sacrifice.

### Sacrifice and Tissue Processing

Animals in the acute permeability group were sacrificed immediately after injury. Under a surgical plane of anesthesia, rats were transcardially perfused using chilled heparinized saline starting at 5 minutes after injury, followed by chilled 4% paraformaldehyde (PFA; Sigma, St. Louis, MO) 8-9 minutes after injury. Heads remained in PFA overnight before brain extraction the following day. Brains were blocked in the coronal plane at 4 mm intervals and transferred to 30% sucrose (Sigma, St. Louis, MO) until saturated. The blocks then were placed in OCT (Fisher Healthcare, Houston, TX), flash frozen in isopentane (Fisher Scientific, Waltham, MA), and stored at −80° C. Twenty micron-thick sections were cut coronally on a cryostat (Leica Biosystems, Buffalo Grove, IL).

Animals in the 7 day survival group were sacrificed 1 week after injury. Animals were anesthetized with isoflurane and transcardially perfused as described above. After extraction the following day, brains were blocked in the coronal plane at 2 mm intervals and processed through paraffin. Eight-micron thick sections were cut coronally on a microtome (Thermo Scientific, Waltham, MA).

### Immunohistochemistry and Imaging

Mounted sections of frozen tissue from animals in the acute permeability group were washed in phosphate buffered saline (PBS) and blocked in 4% normal horse serum (NETS; Sigma, St. Louis, MO) with 0.3% Triton X-100 (Sigma, St. Louis, MO) for 1 h at room temperature. Slices were incubated with primary antibodies in blocking solution overnight at 4° C. Primary antibodies included goat anti-glial fibrillary acid protein (GFAP; 1:1000, Abcam, Cambridge, MA) and rabbit anti-ionized calcium binding adaptor molecule 1 (Iba1; 1:1000, Wako, Richmond, VA). After washing, secondary antibodies were applied at a 1:1000 concentration for 2 h at room temperature in blocking solution: donkey anti-rabbit 647 and donkey anti-goat 568 (Invitrogen, Carlsbad, CA). Sections were counterstained with Hoechst (Hoechst 33342, Life Technologies, Carlsbad, CA) before being cover-slipped with Fluoromount-G (Southern Biotech, Birmingham, AL).

Paraffin-embedded tissue from animals survived 7 days after injury was used for fluorescence and hematoxylin and eosin (H&E) staining. For fluorescence staining, slides were dewaxed in citrosolv (Fisher Scientific, Waltham, MA) and rehydrated in ethanol and deionized water. Antigen retrieval was completed in Tris-EDTA buffer pH 8.0 (Sigma, St. Louis, MO) using a microwave pressure cooker. Tissue was blocked in Optimax buffer (Fisher Scientific, Waltham, MA) plus NETS for 30 minutes at room temperature. Primary antibodies were diluted in Optimax and incubated overnight at 4° C. These antibodies included the GFAP and Iba1 described above, as well as chicken anti-NeuN (1:1000, MilliporeSigma, Burlington, MA). Slides were washed with PBS-Tween (Sigma, St. Louis, MO) before incubation with secondary antibodies (same as frozen sections, in addition to donkey anti-chicken 488, 1:1000, Jackson Immuno Research, West Grove, PA) in Optimax plus NHS for 1 h at room temperature. Slides were cover-slipped with Fluoromount-G.

For H&E staining, tissue was dewaxed in xylene (Fisher Scientific, Pittsburgh, PA) and rehydrated in ethanol and de-ionized water. Nuclei were stained with Mayer's Hematoxylin (Fisher Scientific, Pittsburgh, PA) and blued with lithium carbonate (Sigma, St. Louis, MO). Tissue was counterstained with eosin (Fisher Scientific, Pittsburgh, PA). Eosin was differentiated and slides were dehydrated in ethanol before being cleared in xylene and cover-slipped with Cytoseal (Fisher Scientific, Pittsburgh, PA).

Fluorescence images of were acquired with a Keyence BZ-X800 (Keyence, Osaka, Japan) fluorescent microscope using a 20× objective lens. Examination of H&E sections was performed using light microscopy on an Eclipse E600 (Nikon, Tokyo, Japan) and images were acquired using an Aperio Scanscope CS2 (Leica Biosystems, Buffalo Grove, IL).

### Quantification

For the brain lipid analysis, Agilent ChemStation software was used to determine the corrected area of each FAME peak generated from GC/MS. After excluding the internal standard, the percentage of each fatty acid out of the total amount of fatty acids was determined. The percentage of each FAME was then averaged from 4 animals from each diet.

All image quantification was performed in ImageJ/FIJI by a skilled scientist who was blinded as to the tissue experimental group. In the acute permeability group, a global analysis of permeability was conducted by manually quantifying the number of LY+ cells in three regions—the medial cortex (−3.4 mm bregma), hippocampus (−3.4 mm bregma), and substantia nigra (−5.6 mm bregma)—from 3 slides per region, each 100 μm away from each other. This number was put in context with the size of the area quantified and expressed as cells per mm² for the entire area. For the medial cortex, the region was further subdivided into cortical layers, which were identified using Hoechst. In the medial cortex, the intensity of these cells were measured by drawing two perpendicular lines through the soma of each marked cell to obtain an average mean gray value. Two similarly sized lines were also drawn through the tissue immediately adjacent to each cell to obtain an average background intensity value, which was subtracted from each cell's intensity to attain a measure of permeability dye uptake relative to local background for each permeabilized cell, matching previous methodology.

FIJI was also used to determined GFAP and Iba1 density and intensity from the same sections that were used to assess cortical permeability in acute animals, as well as GFAP, Iba1, and NeuN density and intensity from one section per injured 7 day survival animal. To quantify the number of cells, after adjusting the brightness minimum and maximum equally for all images, images were thresholded to produce a binary image, and a watershed function was applied for NeuN and Iba1 in the 7 day survival tissue. Minimum particle sizes and circularity ranges were optimized to each stain in order to accurately count the number of positive cells. Cell count was put in context with the size of the area quantified and expressed as cells per mm² for the entire area. Fluorescence intensity measurements were made of the entire region of interest, as well as several background (stain-negative) regions within the ROI, which were averaged and subtracted from overall region intensity.

In the 7 day survival group, H&E images were used to determine the size of the lesion area of injured animals at two positions: bregma −3.2 to −4, and bregma −5 to −6.2.

### Statistical Analyses

To assess differences in means, one- or two-way analysis of variance (ANOVA) tests with Tukey's multiple comparison tests were performed. The p values are reported as follows: non-significant p>0.05, * p≤0.05, ** p≤0.01, *** p≤0.001, **** p≤0.0001.

For permeability intensity measurements, the data were visualized as frequency distributions and a line of best fit was determined. As the distributions are not Gaussian, even after transformation, an exponential one-phase decay curve produced the best fit. To determine whether diet effected the distribution of intensity measurements, shams and injured animals were analyzed separately. Non-linear regression was used to determine if one equation fit all three diets within each sham or injured grouping using the extra sum-of-squares F test. If not, the extra sum-of-squares F test was run again with two datasets at a time, until all comparisons between groups were made. To account for multiple comparisons, the original cutoff level of significance (p=0.05) was divided by the total number of comparisons (3), to give a new cutoff for significance of p=0.0167.

### Results

Compromised plasma membrane integrity has been suggested by changes in the blood and/or brain levels of various lipids in both animal models and human cases of TBI. As such, the present study explored whether changes in diet (i.e. ingesting specific lipid constituents) can be used to confer this protection. The plasma membrane is primarily made up of lipids, compounds like fats that do not mix well with water. Lipids come in many varieties, and cell membranes are mainly composed of phospholipids, molecules with one end that is made up of a hydrophobic lipid tail and another end that is soluble in water. There are many different types of phospholipids, and the composition of phospholipids in the plasma membrane can alter its structure and properties. For instance, certain types of lipids can make the cell membrane more or less flexible, as well as impact cell signaling.

One class of lipid in particular seems to have neuroprotective effects. Polyunsaturated fatty acids, or PUFAs, include omega-3 fatty acids like docosahexaenoic acids (DHA) and eicosapentaenoic acid (EPA), are known to have anti-inflammatory properties and provide support to neurons (Michael-Titus, A. T. et al., “Omega-3 fatty acids and traumatic neurological injury: From neuroprotection to neuroplasticity?,” Trends Neurosci., 2014, 37:30-38). Many studies have looked at the effects of these compounds on recovery in animal models of TBI. Several groups have examined the effect of pre-treating rats with a diet rich in omega-three fatty acids or DHA alone before injury, and have found that these animals have reduced markers of injury as well as reduced memory impairments after injury compared to animals eating a normal diet (Mills, J. D. et al., “Dietary supplementation with the omega-3 fatty acid docosahexaenoic acid in traumatic brain injury,” Neurosurgery, 2011, 68:474-481; Wu, A. et al., “Dietary omega-3 fatty acids normalize BDNF levels, reduce oxidative damage, and counteract learning disability after traumatic brain injury in rats,” J. Neurotrauma, 2004, 21:1457-1467). Positive effects on markers of injury and behavioral impairments have also been seen when rats are given DHA in their diets exclusively post-injury (Bailes, J. E. et al., “Docosahexaenoic acid reduces traumatic axonal injury in a rodent head injury model,” J. Neurotrauma, 2010, 27:1617-1624; Wu, A. et al., “Dietary strategy to repair plasma membrane after brain trauma: implications for plasticity and cognition,” Neurorehabil. Neural Repair, 2014, 28:75-84). Separate studies investigating the mechanism of action for DHA have described signaling cascades that are activated to prevent neuronal cell death, reduce oxidative damage, and promote synaptic function (Kim, H.-Y., “Neuroprotection by Docosahexaenoic Acid in Brain Injury,” Mil. Med., 2014, 179:106-111.

In addition to providing anti-inflammatory and trophic support to neurons, PUFAs may also confer neuroprotection by altering the properties of the plasma membrane. When incorporated into other phospholipids, PUFAs make cell membranes more flexible and less rigid (Rawicz, W., et al., “Effect of chain length and unsaturation on elasticity of lipid bilayers,” Biophys. J., 2000, 79:328-339). A study in nematodes examining the cell membranes of touch receptor neurons (that deform to detect sensations) showed that plasma membranes animals lacking the omega-six PUFA arachidonic acid were less flexible than normal (Vasquez, V. et al., “Phospholipids that contain polyunsaturated fatty acids enhance neuronal cell mechanics and touch sensation,” Cell Rep., 2014, 6:70-80). Therefore, plasma membranes enriched with specific lipids or classes of lipids may have distinct physical responses to injury. The flexibility of the plasma membrane plays an important role in the injury response, as stiffer structures are more prone to fail (i.e. break as manifested by micro- or nano-fissures). In fact, studies have shown that when concussion-level strain is applied rapidly to neurons, they develop holes in their plasma membranes (Cullen, D. K., et al., “Trauma-Induced Plasmalemma Disruptions in Three-Dimensional Neural Cultures Are Dependent on Strain Modality and Rate,” J. Neurotrauma, 2011, 28:2219-2233; LaPlaca, M. C. et al., “High rate shear insult delivered to cortical neurons produces heterogeneous membrane permeability alterations,” Annu. Int. Conf. IEEE Eng. Med. Biol.—Proc., 2006, 2384-2387; LaPlaca, M. C. et al., “Plasma membrane damage as a marker of neuronal injury,” Conf. Proc. IEEE Eng. Med. Biol. Soc., 2009, 1113-1116). Alterations in the mechanical properties of the plasma membrane may prevent these tears and therefore prevent and/or attenuate subsequent neuronal dysfunction.

Notably, cellular lipid composition, even in central nervous system tissues, can be easily manipulated via diet (Clandinin, M. et al., “Impact of dietary fatty acid balance on membrane structure and function of neuronal tissues,” Advances in Experimental Medicine and Biology, 1992, 318(4):197-210). Here, the membrane lipid composition in rat brain was altered using three different diets: one supplemented with fish oil to increased PUFAs, one high in SFAs and cholesterol, and one acting as a control diet. After allowing sufficient time for the lipid composition to turn over, animals received lateral fluid percussion injury (FPI) and the effect of diet on plasma membrane permeability and other injury responses were assessed. It was hypothesized that diets rich in elastic PUFAs would decrease injury-induced neuronal plasma membrane permeability, while diets rich in rigid SFAs and cholesterol would increase neuronal membrane permeability.

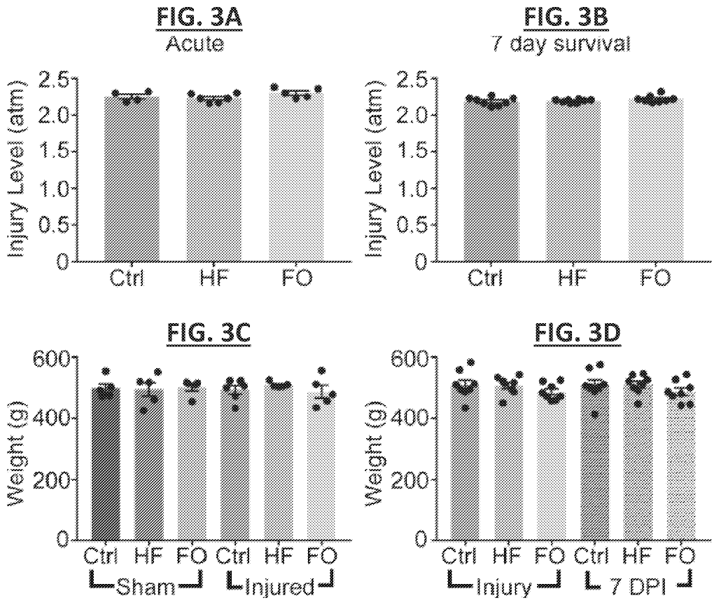

To assess the effects of lipid composition on neuronal plasma membrane permeability and other injury responses, rats were fed one of three different diets: a control diet (Control, abbreviated Ctrl), a diet supplemented with 6% fish oil high in PUFAs (Fish Oil, abbreviated FO), or a diet high in SFAs and cholesterol (High Fat, abbreviated HF) (FIG. 2). After 4 weeks of feeding, one group of animals was used for brain lipid analysis, another group underwent moderate FPI and was immediately examined for acute changes in permeability, while a final group was survived for 7 days post-FPI to investigate other injury outcomes (FIG. 1). There were no differences in weight or injury level between rats in the different diet groups (FIGS. 3A-3D).

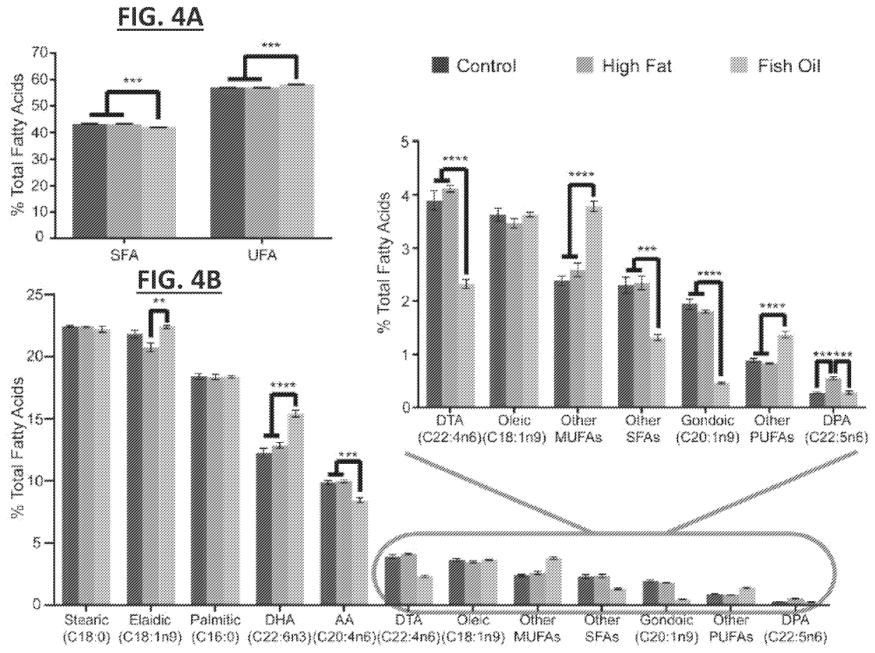

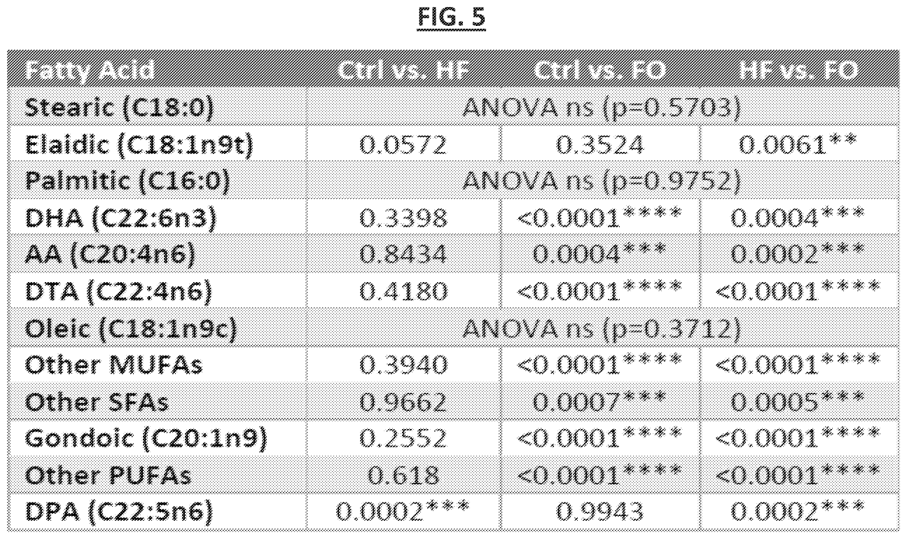

Lipid analysis revealed that the diets altered brain fatty acid composition (FIGS. 4A-4B). As expected, Fish Oil significantly reduced the amount of SFAs (FO vs. Ctrl, p=0.0007; FO vs. HF, p=0.001) and increased the amount of unsaturated fatty acids (FO vs. Ctrl, p=0.0004; FO vs. HF, p=0.0005; FIG. 4A). Examination of specific fatty acids (FIG. 4B, FIG. 5) demonstrated that Fish Oil increased monounsaturated fatty acids and omega-3 PUFAs including docosahexaenoic acid (DHA), while decreasing omega-6 PUFAs such as arachidonic acid, consistent with previous studies (Figueroa, J. D. et al., “Dietary Omega-3 Polyunsaturated Fatty Acids Improve the Neurolipidome and Restore the DHA Status while Promoting Functional Recovery after Experimental Spinal Cord Injury,” Journal of Neurotrauma, 2013, 30(10):853-868; Hals, P.-A. et al., “The time course of erythrocyte membrane fatty acid concentrations during and after treatment of non-human primates with increasing doses of an omega-3 rich phospholipid preparation derived from krill-oil,” Lipids in Health and Disease, 2017, 16(1):16). Surprisingly, High Fat did not increase any of the SFAs measured, but did increase the amount of the omega-6 PUFA docosapentaenoic acid (DPAn6). A review of the literature indicated that this increase in DPAn6 has been observed in animals fed a high fat diet (Levant, B. et al., “Streptozotocin-induced diabetes partially attenuates the effects of a high-fat diet on liver and brain fatty acid composition in mice,” Lipids, 2013, 48(9):939-948; Sharma, S. et al., “High-fat diet transition reduces brain DHA levels associated with altered brain plasticity and behavior,” Scientific Reports, 2012, 2:431). Together, these data show that diet can alter brain fatty acid composition after 4 weeks of feeding.

### Alterations in Plasma Membrane Permeability and Inflammation at Acute Time Points Post-Injury

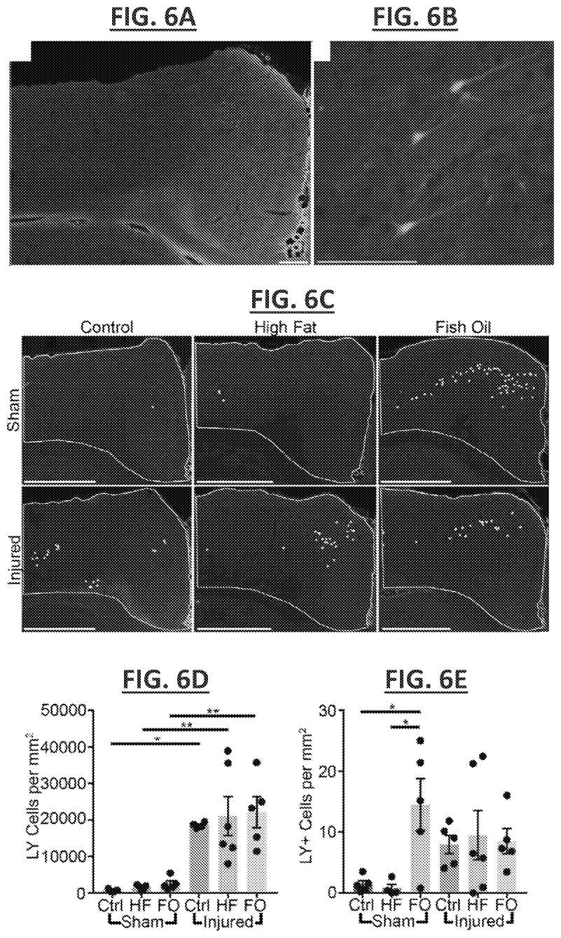

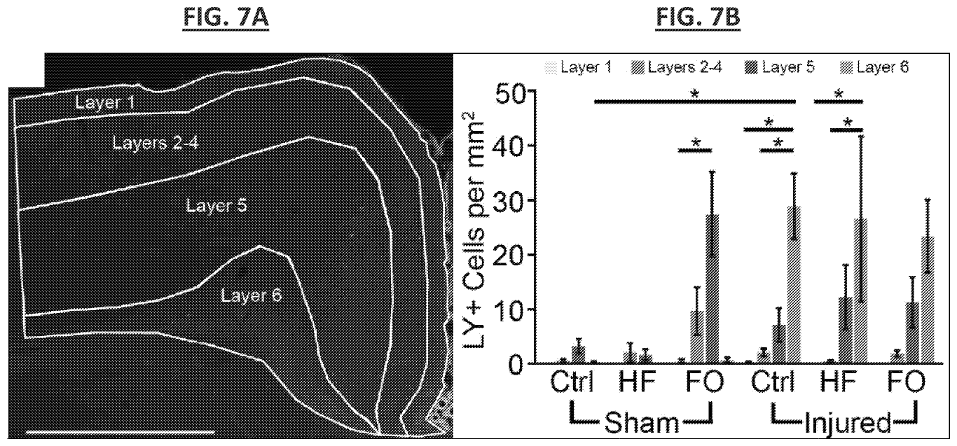

To determine whether plasma membrane lipid composition impacts neuronal permeability, one group of animals underwent FPI two hours after the cell impermeable dye Lucifer Yellow (LY) was delivered to the brain. Five minutes after injury, brains were analyzed using LY to mark cells with loss of membrane integrity. A global analysis of permeabilized neurons throughout the brain revealed a significant increase in the density of mechanoporated neurons due to injury (p<0.0001), but no differences due to diet (p=7101; FIG. 6D). However, a focused analysis of the cerebral cortex did uncover diet-induced alterations in neuronal permeability. At the site of injury, no permeabilized cells were observed (data not shown), suggesting that these neurons were so damaged that they were unable to reseal to trap the dye inside them at this short time point after injury. This observation is likely indicative of acute necrotic cell death at the direct site of injury, as has been observed in other work examining mechanoporation (Farkas, O. et al., “Mechanoporation Induced by Diffuse Traumatic Brain Injury: An Irreversible or Reversible Response to Injury?,” Journal of Neuroscience, 2006, 26(12):3130-3140.). However, the medial cortex adjacent to the injury site did display altered permeability (FIGS. 6A-6E). Unexpectedly, sham animals fed Fish Oil had a high density of permeabilized cells (FO sham vs. Ctrl sham, p=0.0405; FO sham vs. HF sham, p=0.0433). However, there were no diet-induced differences in permeability among injured animals. Two-way ANOVA revealed a significant effect of diet (p=0.0492) and the interaction term (p=0.0389), but not injury (p=0.2094), likely due to the large number of permeabilized neurons in the Fish Oil sham group. Further analysis of the location of the permeabilized cells demonstrated that the increased numbers of LY+ cells in Fish Oil sham animals were mainly localized to layer 5, while injury increased mechanoporation in layer 6 (FIGS. 7A-7B).

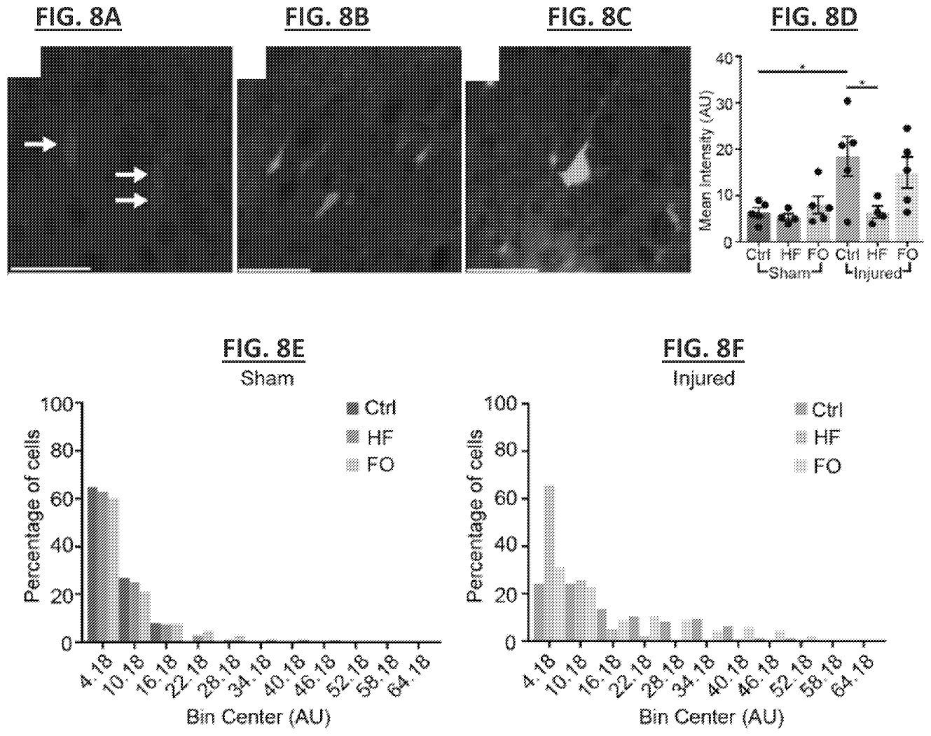

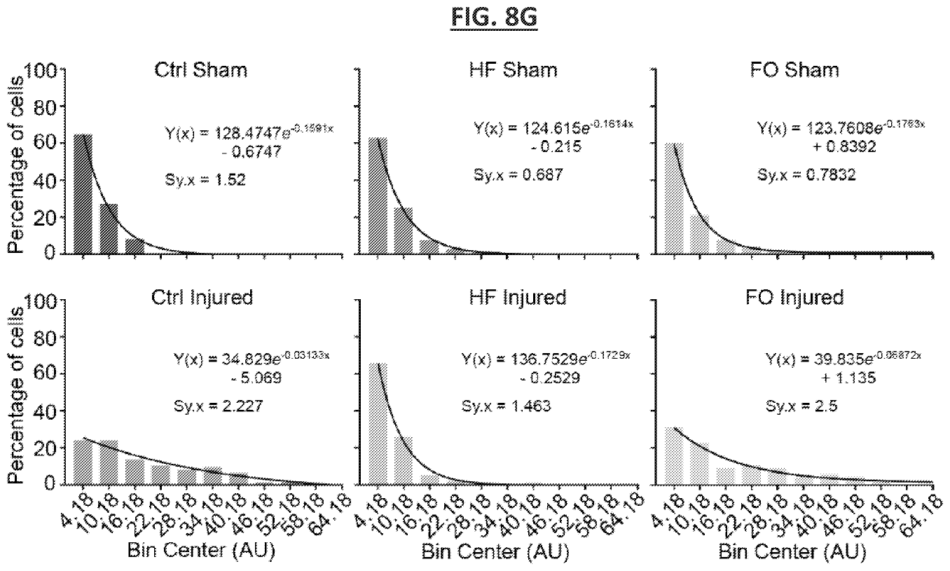

Although injured animals fed each diet did not display differences in the density of permeabilized neurons in the cortex, there were differences in the intensity of dye uptake for individual permeabilized neurons (FIGS. 8A-8G). The extent of dye uptake may be proportional to the extent of plasma membrane damage the neuron has undergone—for neurons that re-seal after injury—with low intensity suggestive of small disruptions that closed quickly, while high intensity could signal larger pores open for longer (Cullen, D. K. et al., “Trauma-Induced Plasmalemma Disruptions in Three-Dimensional Neural Cultures Are Dependent on Strain Modality and Rate,” Journal of Neurotrauma, 2011, 28:2219-2233). For average neuronal intensity of intracellular LY, a two-way ANOVA revealed a significant effect of injury (p=0.0048), a nearly significant effect of diet (p=0.0554), and no significant interaction term (p=0.1501). Multiple comparison analyses showed an injury-induced increase in LY intensity in animals fed the Control diet (p=0.0290), while injured animals fed the High Fat diet had less intense permeability than injured animals fed the Control diet (p=0.0465) (FIG. 8D). An examination of relative frequency distributions of neuronal dye intensity (FIGS. 8E-8G) showed that within the sham group, animals fed Fish Oil had more high intensity cells than animals fed the other diets (FO vs. Ctrl, p=0.0054; FO vs. HF, p=0.0028). Among injured animals, the High Fat diet shifted the distribution towards more low intensity cells compared to the other diets (p=0.0001 for comparisons to both Ctrl and FO). Indeed, the Control and High Fat diets clearly resulted in permeabilized cells with higher dye intensity. Furthermore, because diet altered non-traumatic “incidental” permeability in sham animals, if one were to exclude these incidental low-intensity sham values from each respective injured group, high-intensity neurons from the injured Control and Fish Oil groups would still be present, while no permeabilized neurons would remain in the injured High Fat group. Together, these data show that Fish Oil increased the number and intensity of permeabilized neurons at baseline in sham animals, while the High Fat diet protected against injury-induced increases in the intensity of plasma membrane disruptions.

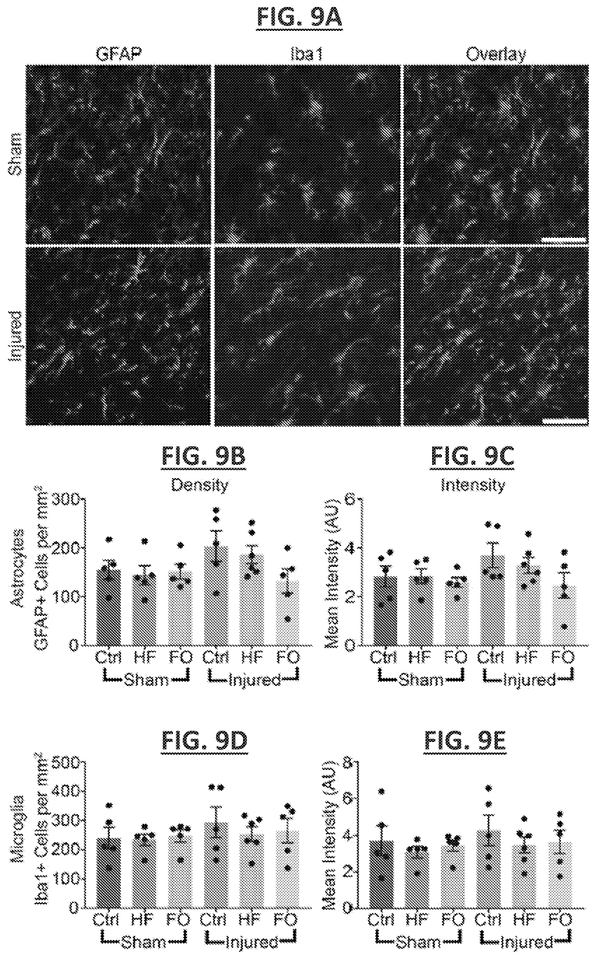

In addition to plasma membrane permeability, inflammation was also examined in the same injury-adjacent medial cortex (FIGS. 9A-9E) There were no significant changes in astrocyte (FIG. 9A-9B) or microglia (FIG. 9C-9D) reactivity (two-way ANOVAs, astrocyte density: injury p=0.1968, diet p=0.2504, interaction p=0.2529; astrocyte intensity: injury p=0.2223, diet p=0.1824, interaction p=0.4618; microglia density: injury p=0.2928, diet p=0.8066, interaction p=0.8406; microglia intensity: injury p=0.4311, diet p=0.4889, interaction p=0.9535). Although fish oil, and its main component DHA, have been reported to confer anti-inflammatory effects via their signaling pathways, it is possible that this 5 minute time point was too acute to observe any changes.

### Neurodegeneration and Inflammation at 7 Days Post-Injury

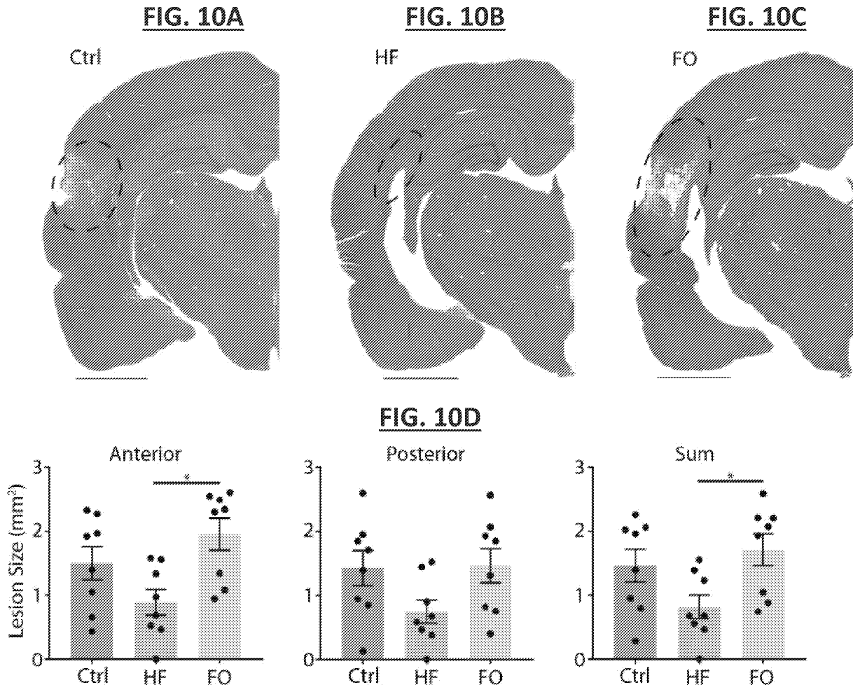

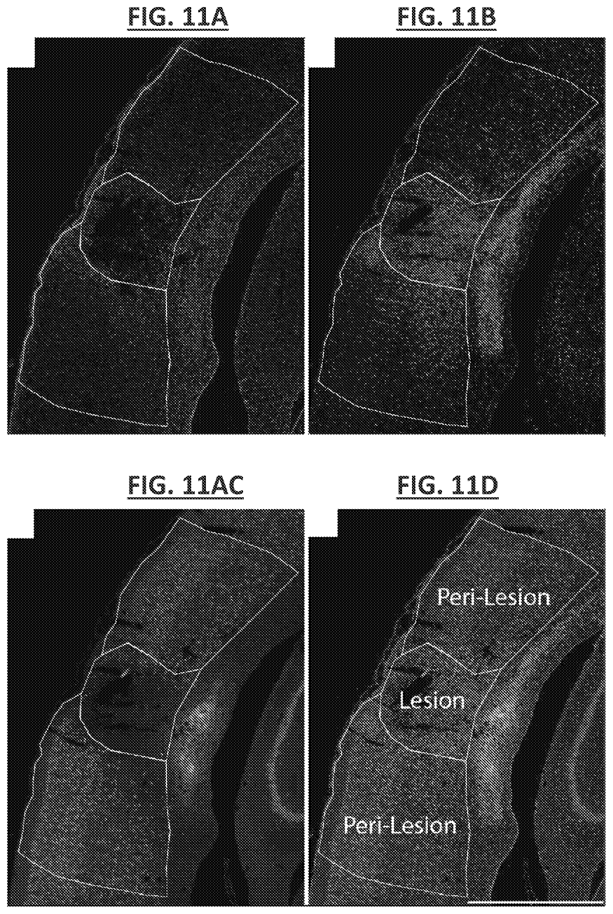

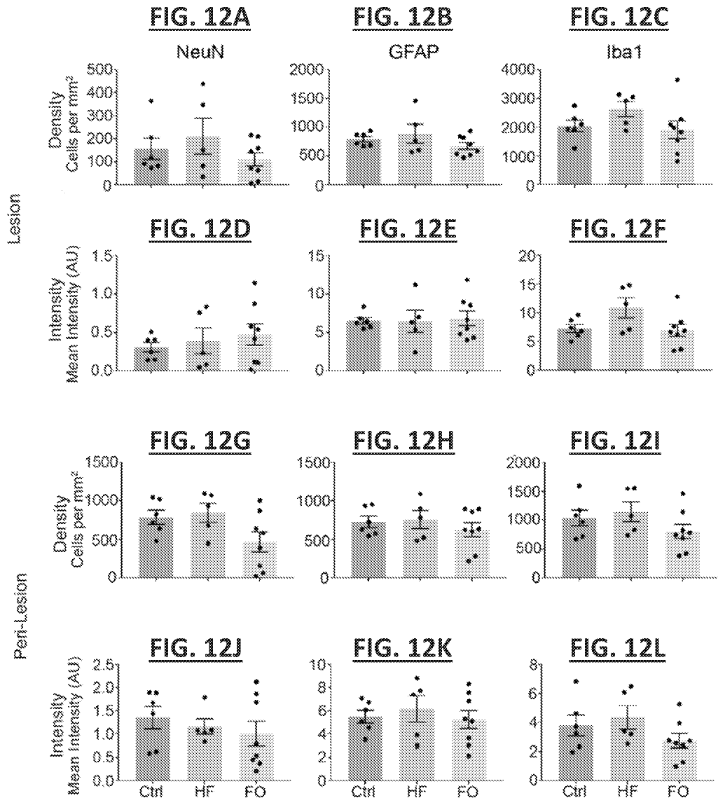

A separate group of animals did not receive LY before FPI and instead were survived for 7 days post-injury to assess longer-term neuropathological consequences of injury as a function of the various diets. First, the extent of degeneration in the cerebral cortex was assessed by measuring the lost tissue in histological sections. Surprisingly, animals fed the High Fat diet had the smallest lesion area (FIGS. 10A-10D; HF vs. FO, p=0.0361 for summed anterior and posterior sections). To further investigate this observation, NeuN expression and inflammation were assessed in the gray matter lesion core and the peri-lesion gray matter 1 mm surrounding the lesion (FIGS. 11A-11D). The gray matter lesion was defined using H&E, and the peri-lesion area consisted of the gray matter 1 mm surrounding the lesion. Animals with only a white matter lesion or no lesion detectable via H&E were excluded. Quantification of NeuN density and intensity in the lesion (FIGS. 12A, 12D) and peri-lesion (FIGS. 12G, 12J) regions revealed a trend for decreased NeuN density in the peri-lesion area of injured animals fed Fish Oil (p=0.0769). While there were no differences in astrocyte reactivity in either region (FIGS. 12B, 12E, 12H, 12K), assessment of microglia density and intensity in the lesion (FIGS. 12C, 12F) and peri-lesion (FIGS. 121, 12L) regions resulted in a trend for increased intensity in the lesion of injured animals fed the High Fat diet (p=0.0734). Together, these data show that animals fed the High Fat diet had smaller lesions while those fed Fish Oil had the largest lesions 7 days after injury, which may be associated with changes in NeuN expression and/or inflammation.

### Overview

The above studies show that diet can be used to alter the susceptibility of the brain to TBI-induced damage. Feeding diets to rats for 4 weeks altered brain lipid composition as follows:

TABLE 1
Alternation in brain lipid composition

| | Control | | |
|---|---|---|---|
| | Diet | High Fat Diet | Fish Oil Diet |
| Saturated Fatty | Baseline | No change from | Decrease in SFAs |
| Acids (SFAs) | | Control | |
| Monosaturated Fatty | | No change from | Increase in MUFAs |
| Acids (MUFAs) | | Control | |
| Polyunsaturated | | Increase in | Decrease in |
| Fatty Acids | | omega-6 | omega-6 PUFAs |
| (PUFAs) | | PUFA DPA | like arachidonic |
| | | | acid, increase in |
| | | | omega-3 PUFAs |
| | | | like DHA |

The present disclosure demonstrates that diets altered the neuronal plasma membrane permeability (FIGS. 6A-6E): the High Fat Diet reduced TBI-induced membrane permeability in the cortex, while the Fish Oil Diet increased the baseline level of membrane permeability. The lesion size 7 days after injury was affected by diet (FIGS. 8A-8G): animals fed the High Fat Diet had the smallest lesion area. Furthermore, the diets altered the brain inflammation (FIGS. 9A-9E): 7 days after injury, animals fed the High Fat Diet had increased microglia reactivity; in contrast, both immediately after injury and to a lesser extent 7 days after injury, animals fed the Fish Oil Diet did not have an increase in astrocyte reactivity. These results indicate that specific lipids are able to alter different aspects of TBI-induced damage. The overall results are shown in Table 2.

TABLE 2
Summary of diet-induced changes to the brain

| | Control Diet | High Fat Diet | Fish Oil Diet |
|---|---|---|---|
| Permeability | Baseline | Decreased after | Increased at |
| | | injury | baseline |
| Lesion Size | | Decreased | Increased |
| Inflammation | | No change/ | Decreased/No |
| | | Increased | change |
| Lipid Peroxidation | | No change | Decreased |

### Selected Discussion

It has been shown herein that diets enriched for specific classes of lipids are able to alter the fatty acid composition of the brain. The Fish Oil diet increased levels of unsaturated fatty acids, especially omega-3 PUFAs like DHA at the expense of omega-6 PUFAs, while the High Fat diet only increased the amount of DPAn6. A more detailed examination of the cortex revealed that the Fish Oil diet increased both the density and intensity of permeabilized cells at baseline in sham animals—indicating that PUFAs influenced passive membrane properties—but had no effect on injury-induced mechanoporation. Surprisingly, injured animals receiving the High Fat diet, although they did not exhibit a reduced cortical density of permeabilized neurons, did display less intense dye uptake in the injury-adjacent cortex, indicating a reduced extent of trauma-induced plasma membrane disruptions. At 7 days post-injury, a separate cohort of animals receiving the High Fat diet had a smaller lesion area than animals receiving the Fish Oil diet. Supporting this finding, animals fed the Fish Oil diet also displayed a trend for fewer NeuN+ cells in the peri-lesion area. At 7 days after injury, a trend was observed for injured animals fed the High Fat diet to display increased microglia intensity in the lesion core.

The present results show that animals fed the High Fat diet had a reduced extent of plasma membrane damage at the time of injury, as well as reduced degeneration 7 days post-injury. Based on this data, the initial hypothesis that diets rich in elastic PUFAs would decrease injury-induced plasma membrane permeability, while diets rich in rigid SFAs and cholesterol would increase permeability, is not supported.

The idea that less intense intracellular dye uptake signifies a reduced extent of plasma membrane damage has been suggested by prior in vitro work monitoring cell survival after cultures were subjected to controlled strain fields resulting in different levels of permeability. In that study, cell death 48 hours after injury was strongly correlated with the degree of per-cell dye uptake rather than the density of permeabilized cells. Future studies of the High Fat diet and its components will employ cell tracking techniques to follow the fate of permeabilized neurons at later time points, including intermediate time points since some injury responses may have already concluded by 7 days after injury.

The present results were unexpected given the large number of previous studies which have found that fish oil, or its main component, DHA, improve other TBI outcomes such as inflammation, oxidative stress, axonal damage, synaptic plasticity, cell death, lesion size, and/or behavior. However, these studies have typically examined the well-known anti-inflammatory and neuroprotective signaling pathways associated with DHA, as opposed to its contributions to membrane properties. Yet, closer inspection of the physical properties of PUFAs reveal that the same structural characteristic that contributes to increased membrane elasticity—the bulky unsaturated hydrocarbon chain—also contributes to increased membrane permeability (Ehringer, W. et al., “A comparison of the effects of linolenic (18:3n3) and docosahexaenoic (22:6n3) acids on phospholipid bilayers, Chemistry and Physics of Lipids, 1990, 54:79-88; Rawicz, W. et al., “Effect of chain length and unsaturation on elasticity of lipid bilayers,” Biophysical Journal, 2000, 79(1):328-339). Indeed, the longer and more unsaturated the hydrocarbon chain, the greater the permeability of the plasma membrane. It is therefore interesting that the only fatty acid that was significantly changed in animals fed the High Fat diet was omega-6 docosapentaenoic acid (DPAn6), which is also a long-chain PUFA with 22 carbons but with one less double bond than DHA. While not wishing to be limited by theory, this slight difference in unsaturation may allow DPAn6 to increase plasma membrane elasticity without compromising the permeability as much as DHA. Indeed, an investigation of membranes containing DHA or DPAn6 revealed no difference in the elastic bending modulus between the two lipids, but did show differences in the packing of hydrocarbon chains (Eldho, N. V. et al., “Polyunsaturated docosahexaenoic vs docosapentaenoic acid—Differences in lipid matrix properties from the loss of one double bond,” Journal of the American Chemical Society, 2003, 125(21):6409-6421), which could influence permeability.

Another possible explanation for the differences in the benefits of fish oil in the present study compared to previous studies could be the timing of administration. The diets were administered for four weeks prior to injury in order to assess the prophylactic effects of various lipid compositions on membrane properties. While a few other studies have also administered fish oil or DHA pre-injury (Mills, J. D. et al., “Dietary supplementation with the omega-3 fatty acid docosahexaenoic acid in traumatic brain injury,” Neurosurgery, 2011, 68(2):474-481; Wang, T. et al., “Effect of fish oil supplementation in a rat model of multiple mild traumatic brain injuries,” Restorative Neurology and Neuroscience, 2013, 31(5):647-659; Wu, Aiguo et al., “Dietary omega-3 fatty acids normalize BDNF levels, reduce oxidative damage, and counteract learning disability after traumatic brain injury in rats,” Journal of Neurotrauma, 2004, 21(10):1457-1467), most have administered post-injury. Although not wishing to be limited by theory, it is hypothesized that, when administered prophylactically, the structural effects of DHA on membrane properties dominate over neuroprotective signaling pathways acutely after injury. When administered post-injury, perhaps the neuroprotective signaling pathways dominate the effects of DHA, as the structural effects are less important at this time. Future studies will be performed to investigate the effects of DHA or fish oil given after an initial injury but before a second injury.

Alternatively, it is possible that fish oil or DHA simply do not improve TBI outcomes. The present data showing trends for a lack of microglial response in injured animals fed Fish Oil at the 7 day time point may suggest that this lack of inflammatory response is harmful and contributes to the increased lesion size. In other animal studies, PUFAs were found to be detrimental in a cerebral ischemia/reperfusion model (Yang, D. Y. et al., “Detrimental effects of post-treatment with fatty acids on brain injury in ischemic rats,” NeuroToxicology, 2007, 28(6):1220-1229). In humans, there have been no published clinical trials, although case studies have described positive effects of fish oil administration after severe TBI (Bailes, J. E. et al., “Omega-3 fatty acid supplementation in severe brain trauma: case for a large multicenter trial,” Journal of Neurosurgery JNS, 2020, 133(2):598-602; Lewis, M. et al., “Therapeutic use of omega-3 fatty acids in severe head trauma,” American Journal of Emergency Medicine, 2013, 31(1):8-11; Sears, B. et al. “Therapeutic uses of high-dose omega-3 fatty acids to treat comatose patients with severe brain injury,” PharmaNutrition, 2013, 1(3):86-89). However, in one small study of collegiate football players that reported a benefit of prophylactic fish oil supplementation in terms of attenuation of elevations in serum neurofilament light (NFL; a biomarker of axonal injury), participants receiving the highest doses of DHA actually sustained the most concussions (Oliver, J. M. et al., “Effect of docosahexaenoic acid on a biomarker of head trauma in American Football,” Medicine and Science in Sports and Exercise, 2016, 48(6):974-982; Trojian, T. J. et al., “Nutritional Supplements for the Treatment and Prevention of Sports-Related Concussion—Evidence Still Lacking,” Current Sports Medicine Reports, 2017, 16(4):247-255). It is therefore possible that rodent injury mechanisms may not translate to human injury, or the timing of administration and dosing of DHA must be carefully calibrated in order to be beneficial in humans. Indeed, DHA is an anticoagulant and could contribute to brain hemorrhage after injury in individuals taking blood thinners (Gross, B. W. et al., “Omega-3 Fatty Acid Supplementation and Warfarin: A Lethal Combination in Traumatic Brain Injury,” Journal of Trauma Nursing, 2017, 24(1):15-18).

While the effects of fish oil or DHA have received much attention, the impact of diets high in saturated fats on TBI outcomes are less well-studied. The results clearly showed a beneficial effect of the High Fat diet. However, in other studies a high fat high sucrose diet given 4 to 8 weeks before either FPI or controlled cortical impact in rats negatively affected TBI outcomes such as cortical tissue loss, activation of protective signaling pathways, and behavior (Hoane, M. R. et al., “The effects of a high-fat sucrose diet on functional outcome following cortical contusion injury in the rat,” Behavioural Brain Research, 2011,223(1):119-124; Wu, A. et al., “A saturated-fat diet aggravates the outcome of traumatic brain injury on hippocampal plasticity and cognitive function by reducing brain-derived neurotrophic factor,” Neuroscience, 2003, 119(2):365-375). Interestingly, a weigh-drop study indicated that the effect of a high fat high sucrose diet on a variety of behaviors may depend on sex and time of injury, with the high fat diet actually improving the performance of injured animals in some cases (Mychasiuk, R. et al., “Diet, age, and prior injury status differentially alter behavioral outcomes following concussion in rats,” Neurobiology of Disease, 2015, 73:1-11). It is important to note that the precise composition of the high fat diets differed among these studies, which may have impacted which lipid constituents were affected. In the present study, the High Fat diet only significantly altered the levels of DPAn6 (out of the panel of lipids measured). Though initially surprising as it was expected that saturated fatty acids would increase and PUFAs would decrease, this result has been observed after administration of other high fat diets as well (Levant, B. et al., “Streptozotocin-induced diabetes partially attenuates the effects of a high-fat diet on liver and brain fatty acid composition in mice,” Lipids, 2013, 48(9):939-948; Sharma, S. et al., “High-fat diet transition reduces brain DHA levels associated with altered brain plasticity and behavior,” Scientific Reports, 2012, 2:431). DPAn6 specifically may be key to the beneficial effects we observed with the High Fat diet. Investigating the properties of this under-studied lipid will be important since the complete high fat diet employed herein is often utilized in models of atherosclerosis and obesity, and would not be suitable for long-term prophylactic use as it would lead to a host of other health problems. Alternatively, other components of the High Fat diet that were not measured could have contributed to its effect on permeability. The primary source of lipids in the diet was milkfat, which contains sphingolipids like sphingomyelin. Interestingly, computational modeling has shown that sphingomyelin decreases membrane permeability in molecular dynamics simulations at equilibrium and under deformation (Saeedimasine, M. et al., “Role of lipid composition on the structural and mechanical features of axonal membranes: a molecular simulation study,” Scientific Reports, 2019, 9:8000). Further studies are necessary to dissect the various components of our High Fat diet to determine the constituent(s) exhibiting the maximal effect on membrane responses and subsequent degeneration following trauma.

Going forward, it will be important to explore the effects of the High Fat diet constituents like DPAn6 when administered pre-injury to modulate membrane properties, as well as to examine whether the elasticity of specific cells and/or whole tissue is affected by diet-induced lipid composition changes. Importantly, however, membrane elasticity may only partially contribute to susceptibility to trauma-induced alterations in membrane permeability. Other factors such as neuronal morphology, complexity of neurite outgrowth, transmembrane proteins, and cell-cell or cell-matrix adhesions (the latter two of which may be influenced by membrane composition) may affect the transfer of strain to cells. Future studies will more broadly examine the effects of neuronal lipid composition on these other components affecting pathological mechanosensation.

Additional studies will determine whether elements of the High Fat diet show any post-injury administration effects on neuroprotective signaling pathways. Very few studies have examined DPA or its metabolites, but the few that do exist have shown anti-inflammatory effects for DPAn6 both in vitro and in vivo, though not in TBI models (Chiu, C.-Y. et al., “Omega-6 docosapentaenoic acid-derived resolvins and 17-hydroxydocosahexaenoic acid modulate macrophage function and alleviate experimental colitis,” Inflammation Research: Official Journal of the European Histamine Research Society, 2012, 61(9):967-976; Dangi, B. et al., “Biogenic synthesis, purification, and chemical characterization of anti-inflammatory resolvins derived from docosapentaenoic acid (DPAn-6),” The Journal of Biological Chemistry, 2009, 284(22):14744-14759). It will also be crucial to investigate outcomes other than immediate plasma membrane permeability, such as cell death and inflammation, as well as changes in cell or whole animal function, at time points later than the ones examined here.

Any additional studies will employ larger sample sizes in an attempt to overcome the apparent inherent variability of dietary modifications superimposed over variability in injury outcomes. The present study was limited by small sample sizes (n=5-6 per group for acute permeability, n=8 per group for 7d survival) and large within-group variability.

Lastly, any future studies assessing the effects of subtle changes in lipid composition on membrane permeability will use additional complimentary markers beyond LY, such as those that bind to intracellular substrates, thus capturing non-resealed cells and potentially allowing cell tracking post-injury [e.g., propidium iodide, ethidium homodimer], as well as larger permeability markers that will likely not be sensitive to minor changes in passive membrane permeability such as larger dextrans. The increase in permeability seen in the Fish Oil sham group—suggesting changes in passive membrane permeability—may indicate that LY is be too sensitive to changes in baseline membrane properties to use as a stand-alone marker of plasma membrane disruptions due to trauma. In traditional studies of trauma-induced membrane permeability, the charge and small size (457 Da) of LY, among other properties, may contribute to the sensitivity of the dye to capture the greatest extent of neurons initially permeabilized by trauma and subsequently resealing.

In conclusion, the present studies have shown that diets enriched with specific types of lipids can alter the fatty acid composition of the brain. A Fish Oil diet that increased omega-3 PUFAs such as DHA resulted in greater baseline plasma membrane permeability, while a High Fat diet that increased DPAn6 resulted in less intense injury-induced plasma membrane permeability. Animals receiving the High Fat diet also demonstrated a smaller lesion area 7 days after injury. These results demonstrate proof-of-concept that diets can modulate acute injury responses, and suggest that prophylactic administration of DPAn6, or other components of the High Fat diet, may be protective against TBI.

### Enumerated Embodiments

The following enumerated embodiments are provided, the numbering of which is not to be construed as designating levels of importance.

Embodiment 1 provides a method of reducing a brain injury in a subject having an elevated risk of a traumatic brain injury (TBI) or concussion, the method comprising: prophylactically administering to the subject a composition comprising a therapeutically effective amount of a polyunsaturated fatty acid.

Embodiment 2 provides the method of embodiment 1, wherein the composition comprising the polyunsaturated fatty acid is encapsulated in a lipid nanoparticle.

Embodiment 3 provides the method of embodiment 1 or 2, wherein the polyunsaturated fatty acid is an omega-6 polyunsaturated fatty acid.

Embodiment 4 provides the method of embodiment 3, wherein the omega-6 polyunsaturated fatty acid is omega-6 docosapentaenoic acid.

Embodiment 5 provides the method of embodiments 1-4, wherein the composition further comprises cholesterol, a triglyceride, a sphingolipid, or a combination thereof.

Embodiment 6 provides the method of embodiment 5, wherein the sphingolipid is a sphingomyelin.

Embodiment 7 provides the method of embodiment 5, wherein the composition comprises an ester derived from glycerol and three fatty acids selected from the group consisting of myristic acid, palmitic acid, stearic acid, palmitoleic acid, oleic acid, linoleic acid, α-linoleic acid, vaccenic acid, and combinations thereof.

Embodiment 8 provides the method of embodiments 2-7, wherein the lipid nanoparticle comprises an omega-6 polyunsaturated fatty acid, cholesterol, a sphingomyelin, or a combination thereof.

Embodiment 9 provides the method of embodiment 8, wherein the omega-6 polyunsaturated fatty acid is omega-6 docosapentaenoic acid.

Embodiment 10 provides the method of any one of embodiments 1-9, wherein the method further comprises the step of continuing to administer to the subject the composition comprising a therapeutically effective amount of a polyunsaturated fatty acid after the subject has suffered a TBI or concussion.

Embodiment 11 provides the method of embodiments 1-10, wherein the composition is orally administered to the subject.

Embodiment 12 provides the method of embodiments 1-11, wherein the method reduces formation of micro- or nano-sized tears in a plasma membrane of a brain cell of the subject formed during a concussion or TBI primary injury.

Embodiment 13 provides a sports drink comprising a polyunsaturated fatty acid composition encapsulated in a lipid nanoparticle, wherein the sports drink further comprises sugar and an electrolyte.

Embodiment 14 provides the sports drink of embodiment 13, wherein the polyunsaturated fatty acid is an omega-6 polyunsaturated fatty acid.

Embodiment 15 provides the sports drink of embodiment 13, wherein the omega-6 polyunsaturated fatty acid is omega-6 docosapentaenoic acid.

Embodiment 16 provides the sports drink of any one of embodiments 13-15, wherein the polyunsaturated fatty acid composition further comprises cholesterol, a triglyceride, a sphingolipid, or a combination thereof.

Embodiment 17 provides the sports drink of embodiment 16, wherein the sphingolipid is a sphingomyelin.

Embodiment 18 provides the sports drink of embodiment 17, wherein the sports drink comprises an ester derived from glycerol and three fatty acids selected from the group consisting of myristic acid, palmitic acid, stearic acid, palmitoleic acid, oleic acid, linoleic acid, α-linoleic acid, vaccenic acid, and combinations thereof.

Embodiment 19 provides the sports drink of embodiments 13-18, wherein the lipid nanoparticle comprises an omega-6 polyunsaturated fatty acid, cholesterol, a sphingomyelin, or a combination thereof.

Embodiment 20 provides the sports drink of embodiment 19, wherein the omega-6 polyunsaturated fatty acid is omega-6 docosapentaenoic acid.

Embodiment 21 provides a dietary supplement comprising a polyunsaturated fatty acid composition encapsulated in a lipid nanoparticle.

Embodiment 22 provides the dietary supplement of embodiment 21, wherein the polyunsaturated fatty acid is an omega-6 polyunsaturated fatty acid.

Embodiment 23 provides the dietary supplement of embodiment 22, wherein the omega-6 polyunsaturated fatty acid is omega-6 docosapentaenoic acid.

Embodiment 24 provides the dietary supplement of embodiments 21-23, wherein the polyunsaturated fatty acid composition further comprises cholesterol, a triglyceride, a sphingolipid, or a combination thereof.

Embodiment 25 provides the dietary supplement of embodiment 24, wherein the sphingolipid is a sphingomyelin.

Embodiment 26 provides the dietary supplement of embodiment 24, wherein the dietary supplement comprises an ester derived from glycerol and three fatty acids selected from the group consisting of myristic acid, palmitic acid, stearic acid, palmitoleic acid, oleic acid, linoleic acid, α-linoleic acid, vaccenic acid, and combinations thereof.

Embodiment 27 provides the dietary supplement of embodiments 21-26, wherein the lipid nanoparticle comprises an omega-6 polyunsaturated fatty acid, cholesterol, a sphingomyelin, or a combination thereof.

Embodiment 28 provides the dietary supplement of embodiment 27, wherein the omega-6 polyunsaturated fatty acid is omega-6 docosapentaenoic acid.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of reducing a brain injury in a subject having an elevated risk of a traumatic brain injury (TBI) or concussion, the method comprising: prophylactically administering to the subject a composition comprising a therapeutically effective amount of a polyunsaturated fatty acid.

2. The method of claim 1, wherein the composition comprising the polyunsaturated fatty acid is encapsulated in a lipid nanoparticle.

3. The method of claim 1, wherein the polyunsaturated fatty acid is an omega-6 polyunsaturated fatty acid.

4. The method of claim 3, wherein the omega-6 polyunsaturated fatty acid is omega-6 docosapentaenoic acid.

5. The method of claim 1, wherein the composition further comprises cholesterol, a triglyceride, a sphingolipid, or a combination thereof.

6. The method of claim 5, wherein the sphingolipid is a sphingomyelin.

7. The method of claim 5, wherein the composition comprises an ester derived from glycerol and three fatty acids selected from the group consisting of myristic acid, palmitic acid, stearic acid, palmitoleic acid, oleic acid, linoleic acid, α-linoleic acid, vaccenic acid, and combinations thereof.

8. The method of claim 2, wherein the lipid nanoparticle comprises an omega-6 polyunsaturated fatty acid, cholesterol, a sphingomyelin, or a combination thereof.

9. The method of claim 8, wherein the omega-6 polyunsaturated fatty acid is omega-6 docosapentaenoic acid.

10. The method of claim 1, wherein the method further comprises the step of continuing to administer to the subject the composition comprising a therapeutically effective amount of a polyunsaturated fatty acid after the subject has suffered a TBI or concussion.

11. The method of claim 1, wherein the composition is orally administered to the subject.

12. The method of claim 1, wherein the method reduces formation of micro- or nano-sized tears in a plasma membrane of a brain cell of the subject formed during a concussion or TBI primary injury.

13. A sports drink comprising a polyunsaturated fatty acid composition encapsulated in a lipid nanoparticle, wherein the sports drink further comprises sugar and an electrolyte.

14. The sports drink of claim 13, wherein the polyunsaturated fatty acid is an omega-6 polyunsaturated fatty acid.

15. The sports drink of claim 13, wherein the omega-6 polyunsaturated fatty acid is omega-6 docosapentaenoic acid.

16. The sports drink of claim 13, wherein the polyunsaturated fatty acid composition further comprises cholesterol, a triglyceride, a sphingolipid, or a combination thereof.

17. The sports drink of claim 16, wherein the sphingolipid is a sphingomyelin.

18. The sports drink of claim 16, wherein the sports drink comprises an ester derived from glycerol and three fatty acids selected from the group consisting of myristic acid, palmitic acid, stearic acid, palmitoleic acid, oleic acid, linoleic acid, α-linoleic acid, vaccenic acid, and combinations thereof.

19. The sports drink of claim 13, wherein the lipid nanoparticle comprises an omega-6 polyunsaturated fatty acid, cholesterol, sphingomyelin, or a combination thereof.

20. The sports drink of claim 19, wherein the omega-6 polyunsaturated fatty acid is omega-6 docosapentaenoic acid.

21. A dietary supplement comprising a polyunsaturated fatty acid composition encapsulated in a lipid nanoparticle.

22. The dietary supplement of claim 21, wherein the polyunsaturated fatty acid is an omega-6 polyunsaturated fatty acid.

23. The dietary supplement of claim 22, wherein the omega-6 polyunsaturated fatty acid is omega-6 docosapentaenoic acid.

24. The dietary supplement of claim 21, wherein the polyunsaturated fatty acid composition further comprises cholesterol, a triglyceride, a sphingolipid, or a combination thereof.

25. The dietary supplement of claim 24, wherein the sphingolipid is a sphingomyelin.

26. The dietary supplement of claim 24, wherein the dietary supplement comprises an ester derived from glycerol and three fatty acids selected from the group consisting of myristic acid, palmitic acid, stearic acid, palmitoleic acid, oleic acid, linoleic acid, α-linoleic acid, vaccenic acid, and combinations thereof.

27. The dietary supplement of claim 21, wherein the lipid nanoparticle comprises an omega-6 polyunsaturated fatty acid, cholesterol, a sphingomyelin, or a combination thereof.

28. The dietary supplement of claim 27, wherein the omega-6 polyunsaturated fatty acid is omega-6 docosapentaenoic acid.
